(19) United States
(12) Patent Application Publication
(10) Pub. No.: US 2025/0318890 A1
(43) Pub. Date: Oct. 16, 2025

(54) TENSION CONTROL UNIT

(71) Applicant: LIVSMED INC., Seongnam-si (KR)

(72) Inventors: Jae Yeong LEE, Seongnam-si (KR); Hee Jin KIM, Seoul (KR); Jwa Nyun KIM, Yongin-si (KR); Chung Hyun LIM, Seoul (KR)

(21) Appl. No.: 19/175,983

(22) Filed: Apr. 10, 2025

(30) Foreign Application Priority Data
Apr. 11, 2024 (KR) 10-2024-0048642

(51) Int. Cl. A61B 34/00; A61B 17/29; A61B 18/00; A61B 18/14

(52) U.S. Cl. CPC A61B 34/71; A61B 18/1445; A61B 2017/2933; A61B 2018/00595; A61B 2018/00601; A61B 2018/1455; A61B 2034/715

(57) ABSTRACT

Provided is a tension control unit for adjusting a tension of a wire in a surgical instrument. The tension control unit includes a body member arranged at one side of an actuation pulley of a manipulation part and connecting the wire to the actuation pulley, a fastening member coupled to one end of the wire, and a bolt member accommodating the fastening member therein and coupled to the body member, wherein the bolt member adjusts the tension of the wire by pulling or releasing the wire according to rotation thereof in a clockwise direction or a counter-clockwise direction.

### CROSS-REFERENCE TO RELATED APPLICATION

This application is based on and claims priority under 35 U.S.C. § 119 to Korean Patent Application No. 10-2024-0048642, filed on Apr. 11, 2024, in the Korean Intellectual Property Office, the disclosure of which is incorporated by reference herein in its entirety.

### BACKGROUND

### 1. Field

The present disclosure relates to a tension control unit, and in particular, to a tension control unit that may be applied to a surgical instrument.

### 2. Description of the Related Art

Surgery denotes a process of curing illness by cutting, incising, or manipulating the skin, the mucosa layer, and other tissues by using a medical instrument. In particular, laparotomy that treats, shapes, or removes an organ by cutting and opening the skin of a surgical site may cause bleeding, side effects, pain of a patient, scar, etc. Therefore, surgery performed by inserting only a medical instrument, e.g., a laparoscope, a surgical instrument, a microscope for microsurgery, etc. after forming a predetermined hole in the skin, or surgery using a robot has been recently considered as an alternative.

A surgical instrument is an instrument for carrying out surgery on a surgical site by manipulating an end tool provided at an end of a shaft that passes through a hole in the skin, manually by a doctor with his/her own hands or by using a robot arm. The end tool provided at the surgical instrument performs a pivoting motion, a gripping motion, a cutting motion, etc. via a predetermined structure.

In addition, a surgical instrument may transfer power to an end tool by using a wire and may control a manipulation performance of the end tool by adjusting a tension of the wire. However, a tension control technique according to the related art occupies a relatively large volume in a mechanical portion and affects the increase in a weight thereof.

The above-mentioned background technology is technical information that the inventor possessed for deriving the present invention or acquired in the process of deriving the present invention, and cannot necessarily be said to be known art disclosed to the general public before filing the application for the present invention.

### SUMMARY

The present disclosure is directed to provide a tension control unit capable of adjusting a grip force of an end tool and a jaw interval by adjusting a tension of a wire, in a surgical instrument which may be operatable manually to be used in laparoscopic surgery or other various surgeries.

Additional aspects will be set forth in part in the description which follows and, in part, will be apparent from the description, or may be learned by practice of the presented embodiments of the disclosure.

According to an embodiment of the present disclosure, a tension control unit for adjusting a tension of a wire in a surgical instrument, includes a body member arranged at one side of an actuation pulley of a manipulation part and connecting the wire to the actuation pulley, a fastening member coupled to one end of the wire, and a bolt member accommodating the fastening member therein and coupled to the body member, wherein the bolt member adjusts the tension of the wire by pulling or releasing the wire according to rotation thereof in a clockwise direction or a counter-clockwise direction.

In an embodiment of the present disclosure, the body member may have a fastening hole formed therein, in which the bolt member and the wire are inserted, and the fastening hole may be formed to pass from one surface to the other surface of the body member.

In an embodiment of the present disclosure, the fastening hole may have a slit having one open side formed in a direction parallel to a lengthwise direction thereof.

In an embodiment of the present disclosure, the slit may be greater than a width of the wire and less than a width of the bolt member.

In an embodiment of the present disclosure, the fastening hole may include a first region capable of accommodating the wire and the bolt member, and a second region capable of accommodating the wire, and the first region may have a region having an inner diameter that is greater than an inner diameter of the second region.

In an embodiment of the present disclosure, the body member may have an opening of the first region formed in one surface thereof and an opening of the second region formed in the other surface thereof.

In an embodiment of the present disclosure, the bolt member may have an accommodation part in which the wire and the fastening member are inserted, and the accommodation part may be formed in a lengthwise direction of the bolt member while passing from one side to the other side of the bolt member.

In an embodiment of the present disclosure, the accommodation part may include a first accommodation part capable of accommodating the wire, and a second accommodation part capable of accommodating the wire and the fastening member, and the second accommodation part may have a region having an inner diameter that is greater than an inner diameter of the first accommodation part.

In an embodiment of the present disclosure, the bolt member may have a stepped part formed on a boundary between the first accommodation part and the second accommodation part, and the fastening member may be restricted from moving toward the second accommodation part due to the stepped part.

In an embodiment of the present disclosure, the accommodation part may have a slit having one open side formed in a direction parallel to a lengthwise direction thereof.

In an embodiment of the present disclosure, the bolt member may be rotatable independently of the wire and the fastening member.

In an embodiment of the present disclosure, the fastening hole may have a screw thread having a preset pitch formed on an inner surface thereof and the bolt member has a screw thread formed on an outer circumference thereof and corresponding to the screw thread of the fastening hole, and the bolt member may be fastened with the fastening hole and a position of the bolt member may be adjustable in a lengthwise direction within the fastening hole according to the rotation of the bolt member.

In an embodiment of the present disclosure, the bolt member may have a D-cut part that is flat and formed in a part of the end portion thereof.

According to an embodiment of the present disclosure, a surgical instrument includes an end tool including one or more rotatable jaws and configured to be rotatable in two or more directions, a manipulation part configured to control rotation of the end tool in the two or more directions, a power transmission part including a wire connected to the manipulation part and transmitting the rotation of the manipulation part to the jaws, and a connection part having one end portion to which the end tool is coupled and the other end portion to which the manipulation part is coupled, and connecting the manipulation part to the end tool, wherein the manipulation part includes an actuation manipulation part comprising an actuation pulley rotating about one shaft and controlling an actuation motion of the end tool, and a tension control unit adjusting a tension of the wire, the tension control unit includes a body member disposed at one side of the actuation pulley of a manipulation part and connecting the wire to the actuation pulley, a fastening member coupled to one end of the wire, and a bolt member accommodating the fastening member therein and coupled to the body member, and the bolt member adjusts the tension of the wire by pulling or releasing the wire according to rotation thereof in a clockwise direction or a counter-clockwise direction.

In an embodiment of the present disclosure, the body member may have a fastening hole formed therein, in which the bolt member and the wire are inserted, and the fastening hole is formed to pass from one surface to the other surface of the body member.

In an embodiment of the present disclosure, the fastening hole may have a slit having one open side formed in a direction parallel to a lengthwise direction thereof.

In an embodiment of the present disclosure, the fastening hole may include a first region capable of accommodating the wire and the bolt member, and a second region capable of accommodating the wire, and the first region may have a region having an inner diameter that is greater than an inner diameter of the second region.

In an embodiment of the present disclosure, the bolt member may have an accommodation part in which the wire and the fastening member are inserted, and the accommodation part may be formed in a lengthwise direction of the bolt member while passing from one side to the other side of the bolt member.

In an embodiment of the present disclosure, the accommodation part may include a first accommodation part capable of accommodating the wire, and a second accommodation part capable of accommodating the wire and the fastening member, and the second accommodation part may have a region having an inner diameter that is greater than an inner diameter of the first accommodation part.

In an embodiment of the present disclosure, the bolt member may be rotatable independently of the wire and the fastening member.

Other aspects, features and advantages other than those described above will become apparent from the following detailed description of the drawings, claims and disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument to which a tension control unit according to an embodiment of the present disclosure is applied;

FIG. 2 is a perspective view illustrating the surgical instrument of FIG. 1;

FIGS. 3 and 4 are perspective views illustrating a manipulation part of the surgical instrument of FIG. 1;

FIG. 5 is a diagram schematically illustrating only a configuration of pulleys and wires constituting joints of the surgical instrument illustrated in FIG. 1;

FIG. 6 is a perspective view illustrating a yaw motion of the surgical instrument of FIG. 2;

FIGS. 7 and 8 are diagrams illustrating a configuration of pulleys and wires, which are related to an actuation motion and a yaw motion of the surgical instrument illustrated in FIG. 1, in detail for each of a first jaw and a second jaw;

FIG. 9 is a perspective view illustrating a pitch motion of the surgical instrument of FIG. 1;

FIGS. 10 and 11 are diagrams illustrating a configuration of pulleys and wires, which are related to a pitch motion of the surgical instrument illustrated in FIG. 1, in detail for each of the first jaw and the second jaw;

FIGS. 12 and 13 are perspective views illustrating an actuation lever motion of the surgical instrument of FIG. 1;

FIGS. 14A and 14B and FIGS. 15A and 15B are diagrams illustrating a movement of a wire during an actuation lever motion of the surgical instrument of FIG. 1;

FIG. 16 is a perspective view illustrating a manipulation part including a tension control unit according to an embodiment of the present disclosure;

FIG. 17 is a plan view of the tension control unit of FIG. 16;

FIG. 18 is a perspective view of the tension control unit and a wire of FIG. 16;

FIG. 19 is a perspective view of a body member in the tension control unit of FIG. 16;

FIG. 20 is a cross-sectional view taken along a line A-A′ of FIG. 19;

FIG. 21 is a cross-sectional view taken along line B-B′ of FIG. 19;

FIG. 22 is a cross-sectional view taken along a line C-C′ of FIG. 19;

FIG. 23 is a perspective view of a bolt member and a wire of FIG. 17;

FIG. 24 is a perspective view of the bolt member from which a wire is removed in FIG. 23;

FIG. 25 is a side-sectional view of the bolt member of FIG. 23; and

FIG. 26 is a perspective view of the bolt member of FIG. 23.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The embodiments will be described below in more detail with reference to the accompanying drawings. Those components that are the same or are in correspondence are rendered the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

In the description, certain detailed explanations of the related art are omitted when it is deemed that they may unnecessarily obscure the essence of the present disclosure.

An expression used in the singular encompasses the expression of the plural, unless it has a clearly different meaning in the context. It will be understood that although the terms “first,” “second,” etc. may be used herein to describe various components, these components should not be limited by these terms. These components are only used to distinguish one component from another. These components are only used to distinguish one component from another.

In the present specification, it is to be understood that the terms such as “including,” “having,” and “comprising” are intended to indicate the existence of the features or components disclosed in the specification, and are not intended to preclude the possibility that one or more other features or components may exist or may be added.

It will be understood that when a unit, region, or component is referred to as being “formed on” another layer, region, or component, it can be directly or indirectly formed on the other layer, region, or component. That is, for example, intervening units, regions, or components may be present.

It will be understood that when an element is referred to as being “connected” or “coupled” to another element, it can be directly connected or coupled to the other element or intervening elements may be present.

Sizes of components in the drawings may be exaggerated for convenience of explanation. In other words, since sizes and thicknesses of components in the drawings are arbitrarily illustrated for convenience of explanation, the following embodiments are not limited thereto.

A tension control unit according to an embodiment of the present disclosure is a device provided to control a tension of a wire in a surgical instrument and may adjust a gripping force of an end tool and a jaw interval in the surgical instrument.

Before describing the tension control unit according to an embodiment of the present disclosure, the surgical instrument to which the tension control unit may be applied is described hereinafter.

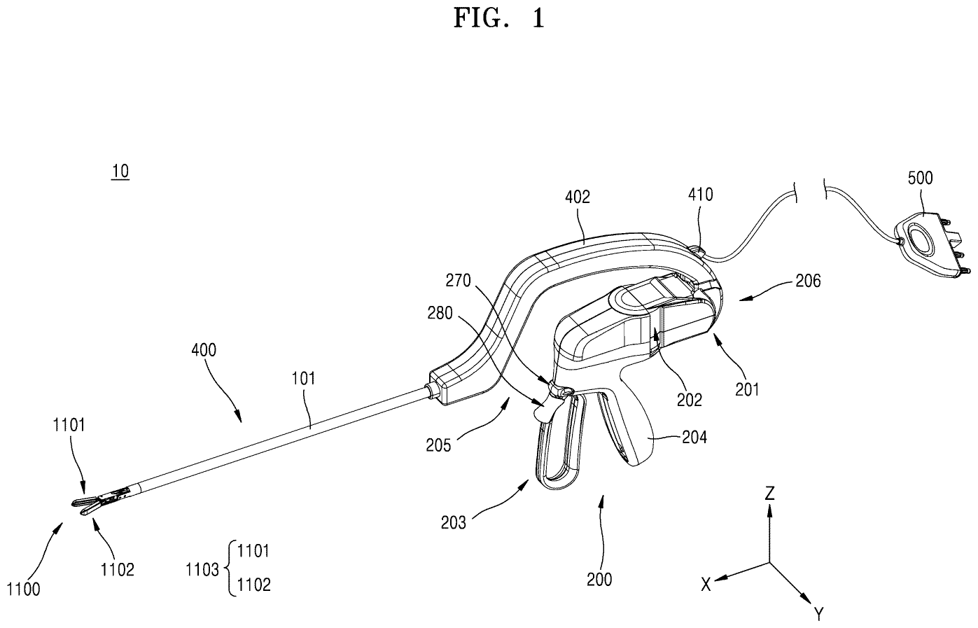

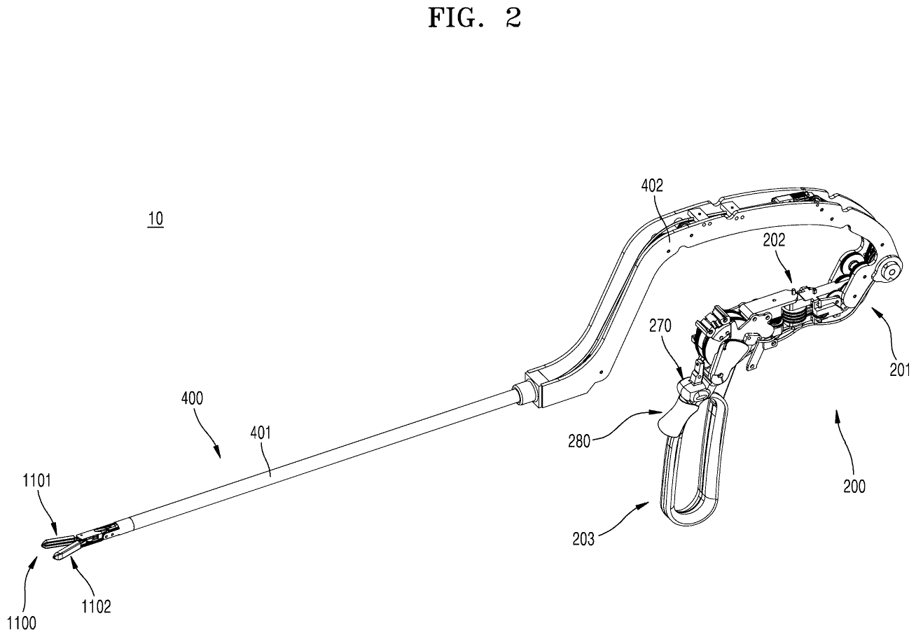

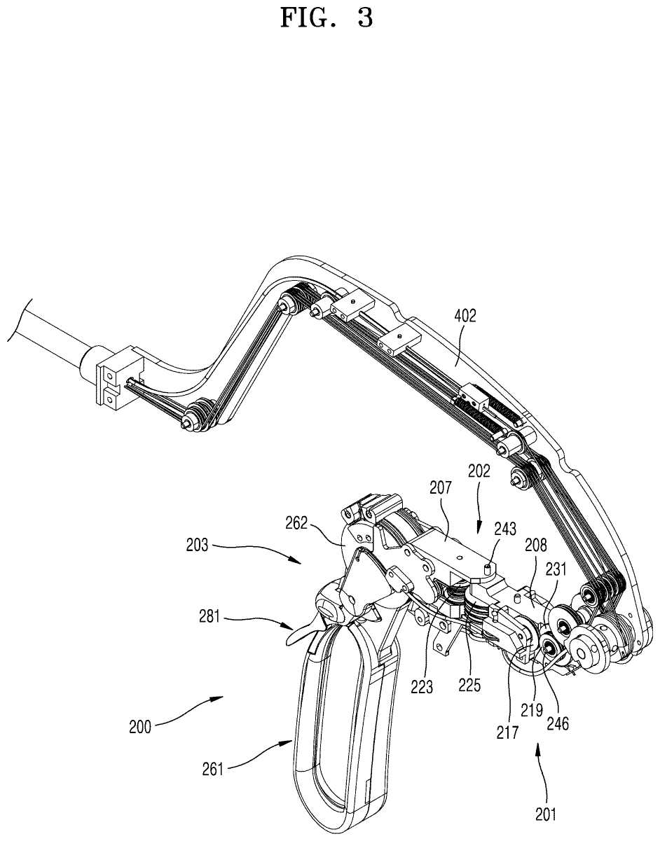

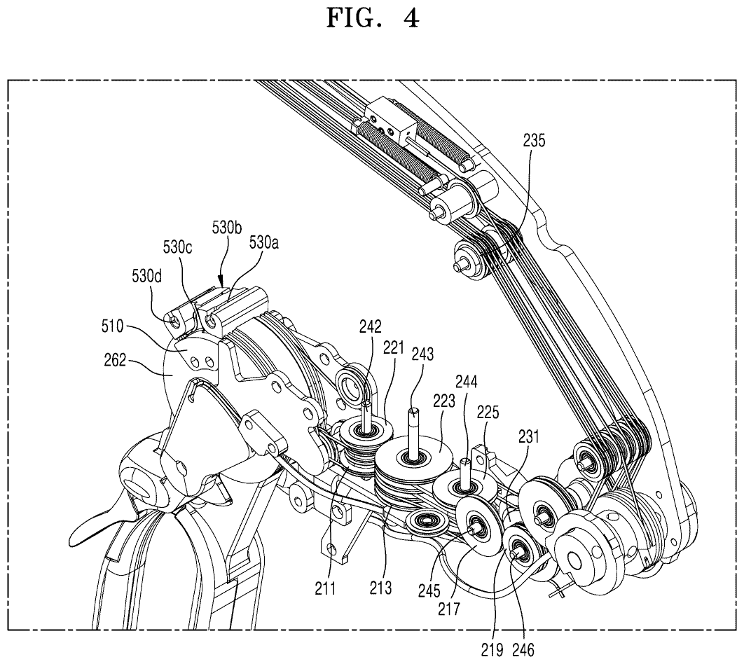

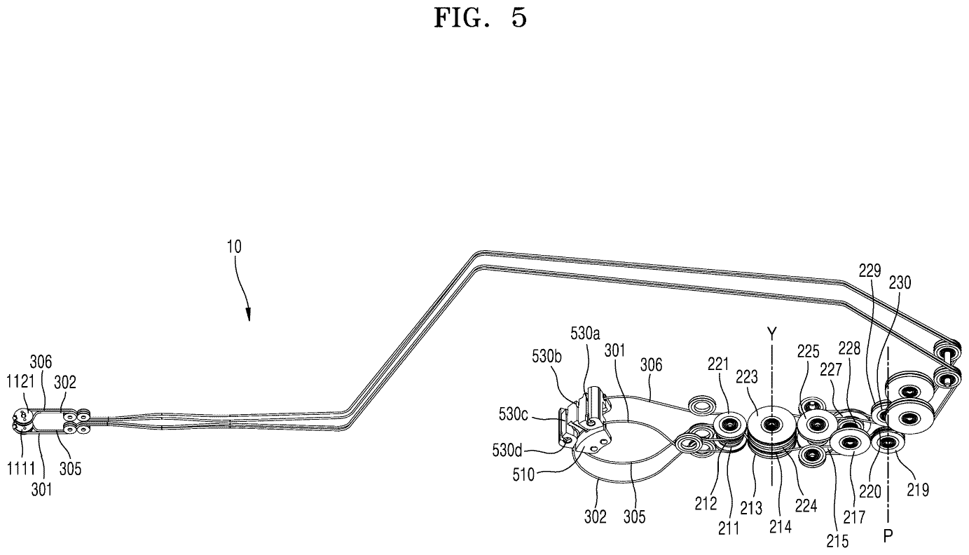

FIG. 1 is a perspective view illustrating a surgical instrument to which a tension control unit according to an embodiment of the present disclosure is applied. FIG. 2 is a perspective view illustrating the surgical instrument of FIG. 1. FIGS. 3 and 4 are perspective views illustrating a manipulation part of the surgical instrument of FIG. 1, and FIG. 5 is a diagram schematically showing a pulley and a wire constituting a joint of the surgical instrument of FIG. 1.

Referring to FIGS. 1 to 5, a surgical instrument 10 according to an embodiment of the present disclosure includes an end tool 1100, a manipulation part 200, a power transmission part 400, and a connection part 400.

Here, the connection part 400 is formed in the shape of a hollow shaft, and one or more wires and electric wires may be accommodated therein. The manipulation part 200 is coupled to one end portion of the connection part 400, the end tool 1100 is coupled to the other end portion of the connection part 400, and thus, the connection part 400 may serve to connect the manipulation part 200 and the end tool 1100. Here, the connection part 400 of the surgical instrument 10 according to the embodiment of the present disclosure includes a straight part 401 and a curved part 402, the straight part 401 is formed at a side of the connection part 400, which is coupled to the end tool 1100, and the curved part 402 is formed at a side of the connection part 400, which is coupled to the manipulation part 200. As such, because the end portion of the connection part 400 at the side of the manipulation part 200 is formed to be curved, a pitch manipulation part 201, a yaw manipulation part 202, and an actuation manipulation part 203 may be formed along an extension line of the end tool 1100 or adjacent to the extension line. From another perspective, it may be said that the pitch manipulation part 201 and the yaw manipulation part 202 are at least partially accommodated in a concave portion formed by the curved part 402. Due to the above-described shape of the curved part 402, the shapes and motions of the manipulation part 200 and the end tool 1100 may be further intuitively matched with each other.

Meanwhile, a plane on which the curved part 402 is formed may be substantially the same as a pitch plane, that is, an XZ plane of FIG. 1. As such, as the curved part 402 is formed on substantially the same plane as the XZ plane, interference with the manipulation part may be reduced. In one or more embodiments, for the intuitively matched motions of the end tool and the manipulation part, any configuration formed on a plane other than the XZ plane may be possible.

Meanwhile, a connector 410 may be formed on the curved part 402. The connector 410 may be connected to an external power source (not shown), and the connector 410 may also be connected to a jaw 1103 via electric wires, and may transmit, to the jaw 1103, electric energy supplied from the external power source (not shown). Here, the connector 410 may be of a bipolar type with two electrodes or a monopolar type with one electrode.

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 1100, which is connected to the interface and inserted into the body of a surgical patient, performs a certain motion, thereby performing surgery. Here, the manipulation part 200 is illustrated in FIG. 1 as being formed in a handle shape that is rotatable while the finger is inserted therein, but the concept of the present disclosure is not limited thereto, and various types of manipulation parts that may be connected to the end tool 1100 and manipulate the end tool 1100 may be possible.

The end tool 1100 is formed on the other end portion of the connection part 400, and performs necessary motions for surgery by being inserted into a surgical site. As an example of the end tool 1100 described above, a pair of jaws 1103 for performing a grip motion may be used as illustrated in FIG. 1. However, the concept of the present disclosure is not limited thereto, and various devices for performing surgery may be used as the end tool 1100. For example, a configuration of a cantilever cautery may also be used as the end tool. The end tool 1100 is connected to the manipulation part 200 via a power transmission part 300 and receives a driving force of the manipulation part 200 through the power transmission part 300, and thus performs a motion necessary for surgery such as gripping, cutting, suturing, or the like.

Here, the end tool 1100 of the surgical instrument 10 according to the embodiment of the present disclosure is formed to be rotatable in one or more directions, for example, the end tool 1100 may be configured to perform a pitch motion around a Y-axis of FIG. 1 and simultaneously perform a yaw motion and an actuation motion around a Z-axis of FIG. 1.

The power transmission part 300 serves to connect the manipulation part 200 to the end tool 1100 and transmit the driving force of the manipulation part 200 to the end tool 1100, and may include a plurality of wires, pulleys, links, sections, gears, or the like.

The end tool 1100, the manipulation part 200, the power transmission part 300, and the like of the surgical instrument 10 of FIG. 1 will be described in detail later.

The power transmission unit 300 of the surgical instrument 10 according to an embodiment of the present disclosure may include a wire 301, a wire 302, a wire 303, a wire 304, a wire 305, a wire 306, and a blade wire 307.

Here, the wires 301 and 305 may be paired to serve as first jaw wires. The wire 302 and the wire 306 may be paired to serve as second jaw wires. Here, elements including the wire 301 and the wire 305 that are the first jaw wires and the wire 302 and the wire 306 that are the second jaw wires may be referred to as jaw wires. In addition, the wire 303 and the wire 304 may be paired to serve as pitch wires.

Also, the power transmission part 300 of the surgical instrument 10 according to an embodiment of the present disclosure may include a fastening member which is coupled to each end portion of each wire in order to couple the wire to a pulley. Here, each fastening member may have various shapes as necessary, such as a ball shape, a tube shape, and the like.

In describing the present disclosure, a part close to a user, that is, a part adjacent to the manipulation part 200, is described as a proximal end, and a part apart from the user, that is, a part adjacent to the end tool 1100, is described as a distal end.

For example, referring to FIG. 2, a part of the end tool 1100, which is adjacent to the manipulation part 200, is defined as a proximal end of the end tool 1100, and a part of the end tool 1100, which is apart from the manipulation part 200, that is, a part adjacent to the end of the end tool 1100, is defined as a distal end of the end tool 1100. From another perspective, the proximal end of the end tool 1100 is described as a part adjacent to the connection part 400 and the distal end of the end tool 1100 is described as a part apart from the connection part 400.

Referring to FIGS. 1 to 4, the manipulation part 200 of the surgical instrument 10 according to the embodiment of the present disclosure includes a first handle 204 that the user may grip, the actuation manipulation part 203 controlling the actuation motion of the end tool 1100, the yaw manipulation part 202 controlling the yaw motion of the end tool 1100, and the pitch manipulation part 201 controlling the pitch motion of the end tool 1100. Here, it may be understood that FIGS. 3 and 4 only show the elements associated with the pitch/yaw/actuation motions of the surgical instrument 10.

In addition, the manipulation part 200 of the surgical instrument 10 further includes a cutting manipulation part 280 performing a cutting motion by controlling movement of a blade 1175 of the end tool 1100, and a sealing manipulation part 270 controlling cauterization by supplying electrical energy to a first electrode 1151 and a second electrode 1152 of the end tool 1100.

The manipulation part 200 may include a pulley 211, a pulley 212, a pulley 213, a pulley 214, a pulley 215, a pulley 217, a pulley 218, a pulley 219, and a pulley 220 related to the rotation movement of the first jaw 1101. Also, the manipulation part 200 may include a pulley 221, a pulley 222, a pulley 223, a pulley 224, a pulley 225, a pulley 227, a pulley 228, a pulley 229, and a pulley 230 related to the rotation movement of the second jaw 1102. Also, the manipulation part 200 may include a pulley 262 related to rotation movements of the first jaw and the second jaw. Also, the manipulation part 200 may include a pulley 231 related to a pitch movement. In addition, the manipulation part 200 may include relay pulleys 235 disposed in the middle of the curved part 402 of the connection part 400.

Here, in the drawings, the pulleys facing each other are formed parallel to each other, but the concept of the present disclosure is not limited thereto, and each of the pulleys may be variously formed at a position and in a size suitable for the configuration of the manipulation part.

Also, the manipulation part 200 according to the embodiment of the present disclosure may include a rotation shaft 241, a rotation shaft 242, a rotation shaft 243, a rotation shaft 244, a rotation shaft 245, and a rotation shaft 246. Here, the rotation shaft 241 may serve as a rotation shaft of the manipulation part, and the rotation shaft 242 may serve as a first yaw sub-rotation shaft of the manipulation part. In addition, the rotation shaft 243 may serve as a yaw main rotation shaft of the manipulation part, and the rotation shaft 244 may serve as a second yaw sub-rotation shaft of the manipulation part. In addition, the rotation shaft 245 may serve as a pitch sub-rotation shaft of the manipulation part, and the rotation shaft 246 may serve as a pitch main rotation shaft of the manipulation part.

The rotation shaft 241, the rotation shaft 242, the rotation shaft 243, the rotation shaft 244, the rotation shaft 245, and the rotation shaft 246 may be sequentially arranged from the distal end 205 toward the proximal end 206 of the manipulation part 200.

Each of the rotation shafts 241, 242, 243, 244, 245, and 246 may be fitted into one or more pulleys, which will be described in detail below.

The pulley 262 may serve as an actuation pulley of the first jaw and the second jaw, and may be referred to as a manipulation part actuation pulley.

The pulley 211 and the pulley 212 may serve as first jaw first yaw sub-pulleys of the manipulation part, and the pulley 221 and the pulley 222 may serve as second jaw first yaw sub-pulleys of the manipulation part, which may be collectively referred as first yaw sub-pulleys of the manipulation part.

The pulley 213 and pulley 214 serve as first jaw yaw main pulleys of the manipulation part, and the pulley 223 and the pulley 224 serve as second jaw yaw main pulleys of the manipulation part, which may be collectively referred to as manipulation part yaw main pulleys.

The pulley 215 serves as a first jaw second yaw sub-pulley of the manipulation part, and the pulley 225 serves as a second jaw second yaw sub-pulley of the manipulation part, which may be collectively referred to as manipulation part second yaw sub-pulleys.

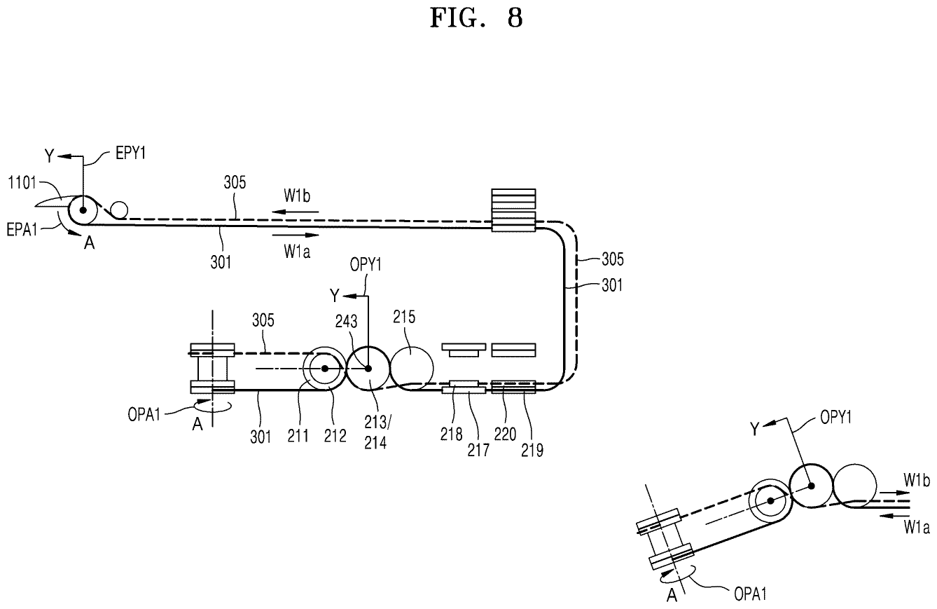

The pulley 217 and the pulley 218 (see FIG. 8) may serve as first jaw pitch sub-pulleys of the manipulation part, and the pulley 227 and the pulley 228 may serve as second jaw pitch sub-pulleys of the manipulation part, which may be collectively referred as pitch sub-pulleys of the manipulation part.

The pulley 219 and the pulley 220 may serve as first jaw pitch main pulleys of the manipulation part, and the pulley 229 and the pulley 230 may serve as second jaw pitch main pulleys of the manipulation part, which may be collectively referred as pitch main pulleys of the manipulation part.

The pulley 231 may serve as a pitch wire main pulley of the manipulation part and may include a pulley (not shown) that serves as a pitch wire sub-pulley of the manipulation part.

The above elements are classified as follows from the perspective of the manipulation part for each movement (pitch/yaw/actuation).

The pitch manipulation part 201 controlling a pitch movement of the end tool 1100 may include the pulley 217, the pulley 218, the pulley 219, the pulley 220, the pulley 227, the pulley 228, the pulley 229, the pulley 230, and the pulley 231. Also, the pitch manipulation part 201 may include the rotation shaft 245 and the rotation shaft 246. Also, the pitch manipulation part 201 may further include a pitch frame 208.

The yaw manipulation part 202 controlling a yaw movement of the end tool 1100 may include the pulley 211, the pulley 211, the pulley 213, the pulley 214, the pulley 215, the pulley 221, the pulley 222, the pulley 223, the pulley 224, and the pulley 225. Also, the yaw manipulation part 202 may include the rotation shaft 242, the rotation shaft 243, and the rotation shaft 244. Also, the yaw manipulation part 202 may further include a yaw frame 207.

An actuation manipulation part 203 controlling an actuation motion of the end tool 1100 may include the pulley 262 and the rotation shaft 241.

Hereinafter, each component of the manipulation part 200 will be described in more detail.

The first handle 204 may be formed to be gripped by a user with the hand, and specifically, may be formed to be gripped by the user by wrapping the first handle 204 with his/her palm. In addition, the actuation manipulation part 203 and the yaw manipulation part 202 are formed on the first handle 204, and the pitch manipulation part 201 is formed at one side of the yaw manipulation part 202. In addition, the other end portion of the pitch manipulation part 201 is connected to the curved part 402 of the connection part 400.

The actuation manipulation part 203 may include an actuation lever 261, the actuation pulley 262, and an actuation reconstruction elastic member 263.

Here, the actuation lever 261 may be formed in a hand ring shape and may act as a second handle.

Here, the rotation shaft 241 that is the actuation rotation shaft may be formed to have a certain angle with respect to an XZ plane where the connection part 400 is formed.

For example, the rotation shaft 241 may be formed parallel to a Y-axis, and when the pitch manipulation part 201 or the yaw manipulation part 202 rotate in this state, a coordinate system of the actuation manipulation part 203 may relatively change. In one or more embodiments, the concept of the present disclosure is not limited thereto, and according to an ergonomic design, the rotation shaft 241 may be formed in various directions to be suitable for the hand structure of the user gripping the actuation manipulation part 203.

In addition, the actuation pulley 262 may be fixedly coupled to the actuation lever 261 or may be formed as one member. Therefore, the actuation pulley 262 may be rotated as well according to the rotation of the actuation lever 261.

Here, the actuation pulley 262 may include one pulley or may include two pulleys that are fixedly coupled to each other.

The yaw manipulation part 202 may include the rotation shaft 242, the rotation shaft 243, the pulley 213 and the pulley 214 that are the first jaw yaw main pulleys of the manipulation part, the pulley 223 and the pulley 224 that are the second jaw yaw main pulleys of the manipulation part, and the yaw frame 207. Also, the yaw manipulation part 202 may further include the pulley 211 and the pulley 212 that are the first jaw first yaw sub-pulleys of the manipulation part and are formed at the sides of the pulley 213 and the pulley 214, and the pulley 221 and the pulley 222 that are the second jaw first yaw sub-pulleys of the manipulation part and formed at the sides of the pulley 223 and the pulley 224. Also, the yaw manipulation part 202 may further include the pulley 215 that is the first jaw second yaw sub-pulley of the manipulation part and is formed at the other sides of the pulley 213 and the pulley 214, and the pulley 225 that is the second jaw second yaw sub-pulley of the manipulation part and formed at the other sides of the pulley 223 and the pulley 224. Here, the pulley 215 and the pulley 225 may be coupled to the pitch frame 208 that is described later.

Here, in the drawings, the yaw manipulation part 202 includes the pulley 213 and the pulley 214, and the pulley 223 and the pulley 224, and the pulley 213 and the pulley 214 and the pulley 223 and the pulley 224 are provided with two pulleys that are formed to face each other and independently rotatable, but the concept of the present disclosure is not limited thereto. That is, one or more pulleys having the same or different diameters may be provided according to the configuration of the yaw manipulation part 202.

In detail, the rotation shaft 242 that is the first yaw sub-rotation shaft of the manipulation part is formed at one side of the actuation manipulation part 203 on the first handle 204, and the rotation shaft 243 that is the yaw main rotation shaft of the manipulation part is formed at one side of the rotation shaft 242. Here, the first handle 204 may be formed to be rotatable about the rotation shaft 243.

Here, the rotation shaft 243 may be formed to have a certain angle with respect to the XY plane where the connection part 400 is formed. For example, the rotation shaft 243 may be formed parallel to the Z-axis, and in this state, when the pitch manipulation part 201 rotates, the coordinate system of the rotation shaft 243 may relatively change as described above. In one or more embodiments, the concept of the present disclosure is not limited thereto, and according to an ergonomic design, the rotation shaft 243 may be formed in various directions to be suitable for the hand structure of the user gripping the manipulation part 200.

In addition, the pulley 213 and the pulley 214, and the pulley 223 and the pulley 224 are coupled to the rotation shaft 243 to be rotatable about the rotation shaft 243. In addition, the pulley 213 and the pulley 214 are wound with the wire 301 or the wire 305 that is the first jaw wire, and the pulley 223 and the pulley 224 may be wound with the wire 302 or the wire 306 that is the second jaw wire. Here, the pulley 213 and the pulley 214, and the pulley 223 and the pulley 224 may be each provided with two pulleys that are formed to face each other and independently rotatable. Therefore, the wire that is wound on and the wire that is wound out may be respectively wound on separate pulleys, and may move without interfering with each other.

The yaw frame 207 rigidly connects the first handle 204, the rotation shaft 242, and the rotation shaft 243, the actuation manipulation part 203 in which the rotation shaft 241 and the actuation pulley 262 are coupled to each other is rigidly connected to the yaw frame 207 directly or via a relay member, so that the first handle 204, the yaw manipulation part 202, and the actuation manipulation part 203 may be integrally yaw-rotated about the rotation shaft 243.

The pitch manipulation part 201 may include the rotation shaft 246, the pulley 219 and the pulley 220 that are the first jaw pitch main pulleys of the manipulation part, the pulley 229 and the pulley 230 that are the second jaw pitch main pulleys of the manipulation part, and the pitch frame 208. Also, the pitch manipulation part 201 may further include the rotation shaft 245, the pulley 217 and the pulley 218 that are the first jaw pitch sub-pulleys of the manipulation part and are formed at the sides of the pulley 219 and the pulley 220, and the pulley 227 and the pulley 228 that are the second jaw pitch sub-pulleys of the manipulation part and are formed at the sides of the pulley 229 and the pulley 230. The pitch manipulation part 201 may be connected to the curved part 402 of the connection part 400 through the rotation shaft 246.

In detail, the pitch frame 208 is a base frame of the pitch manipulation part 201 and has one end portion to which the rotation shaft 243 is rotatably coupled. That is, the yaw frame 207 is formed to be rotatable about the rotation shaft 243 with respect to the pitch frame 208.

As described above, the yaw frame 207 connects the first handle 204, the rotation shaft 243, the rotation shaft 241, and the rotation shaft 242, and because the yaw frame 207 is axially coupled to the pitch frame 208, when the pitch frame 208 pitch-rotates about the rotation shaft 246, the yaw frame 207, the first handle 204, the rotation shaft 241, the rotation shaft 242, and the rotation shaft 243 connected to the pitch frame 208 are pitch rotated as well. That is, when the pitch manipulation part 201 rotates about the rotation shaft 246, the actuation manipulation part 203 and the yaw manipulation part 202 are rotated along with the pitch manipulation part 201. In other words, when the user rotates the first handle 204 about the rotation shaft 246, the actuation manipulation part 203, the yaw manipulation part 202, and the pitch manipulation part 201 are moved along with the first handle 204.

The pulley 219 and pulley 220, and the pulley 229 and pulley 230 are coupled to the rotation shaft 246 so as to be rotatable about the rotation shaft 246 of the pitch frame 208.

Here, the pulley 219 and the pulley 220 may be formed to face each other and to be rotatable independently. Therefore, the wire that is wound in and the wire that is wound out may be respectively wound on separate pulleys, and thus, may operate without interfering with each other. Likewise, the pulley 229 and the pulley 230 may be formed to face each other and to be rotatable independently. Therefore, the wire that is wound in and the wire that is wound out may be respectively wound on separate pulleys, and thus, may operate without interfering with each other.

A connection relationship of the first handle 204 with respect to each of the pitch manipulation part 201, the yaw manipulation part 202, and the actuation manipulation part 203 is summarized as follows. The rotation shaft 241, the rotation shaft 242, the rotation shaft 243, the rotation shaft 244, the rotation shaft 245, and the rotation shaft 246 may be formed above the first handle 204. Here, because the rotation shaft 242 and the rotation shaft 243 are directly formed on the first handle 204, the first handle 204 and the yaw manipulation part 202 may be directly connected to each other. On the contrary, the pitch manipulation part 201 is formed at one side of the yaw manipulation part 202 to be connected to the yaw manipulation part 202, and thus, the pitch manipulation part 201 is not directly connected to the first handle 204, but the pitch manipulation part 201 and the first handle 204 may be indirectly connected to each other via the yaw manipulation part 202. Also, the actuation manipulation part 203 is formed at the other side of the yaw manipulation part 202 to be connected to the yaw manipulation part 202, and thus, actuation manipulation part 203 is not directly connected to the first handle 204, but the actuation manipulation part 203 and the first handle 204 may be configured to be indirectly connected to each other via the yaw manipulation part 202.

Continuing to refer to the drawings, in the surgical instrument 10 according to the embodiment of the present disclosure, the pitch manipulation part 201 and the end tool 1100 may be formed on the same or parallel axis (X-axis). That is, the rotation shaft 246 of the pitch manipulation part 201 is formed at one end portion of the curved part 402 of the connection part 400, and the end tool 1100 is formed at the other end portion of the connection part 400.

In addition, one or more relay pulleys 235 configured to change or guide passages of the wires may be disposed at some parts along the connection part 400, particularly in the curved part 402. As at least some of the wires are wound around the relay pulleys 235 to guide the passages of the wires, these wires may be disposed along a curved shape of the curved part 402.

Here, in the drawings, it is illustrated that the connection part 400 is formed to be curved with a predetermined curvature by having the curved part 402, but the concept of the present disclosure is not limited thereto, and the connection part 400 may be formed linearly or to be curved one or more times as necessary, and even in this case, it may be said that the pitch manipulation part 201 and the end tool 1100 are formed on substantially the same axis or parallel axes. In addition, although FIG. 2 illustrates that each of the pitch manipulation part 201 and the end tool 1100 is formed on an axis parallel to the X-axis, the concept of the present disclosure is not limited thereto, and the pitch manipulation part 201 and the end tool 1100 may be formed on different axes.

The actuation motion, the yaw motion, and the pitch motion according to the embodiment are described as follows.

The actuation motion is as follows.

When the user rotates the actuation lever 261 by using his/her fingers while the finger is inserted in the hand ring formed in the actuation lever 261, the actuation pulley 262 fixedly coupled to the actuation lever 261 is rotated about the rotation shaft 241.

Here, the wire 301 and the wire 305 of which one end portions are fixedly coupled to the pulley 262 to be wound on the pulley 262 and the wire 302 and the wire 306 of which one end portions are fixedly coupled to the pulley 262 to be wound on the pulley 262 are moved according to the rotation of the pulley 262. Here, although the wires 301, 302, 305, and 306 are coupled to one actuation pulley 262, the movement of the wire according to the rotation of the pulley varies depending on the direction of winding each wire on the pulley 262. This will be described in detail later.

In addition, the above rotational force is transmitted to the end tool 1100 via the power transmission part 300, and thus, two jaws 1103 of the end tool 1100 perform the actuation motion.

Here, as described above, the actuation motion denotes a motion of opening or closing the jaws 1101 and 1102 while the two jaws 1101 and 1102 rotate in opposite directions to each other. That is, when the actuation lever 261 of the actuation manipulation part 203 is rotated in a direction close to the first handle 204, the first jaw 1101 rotates in a counter-clockwise direction and the second jaw 1102 rotates in a clockwise direction so that the end tool 1100 is closed. On the contrary, when the actuation lever 261 of the actuation manipulation part 203 is rotated in a direction away from the first handle 204, the first jaw 1101 is rotated in the clockwise direction and the second jaw 1102 is rotated in the counter-clockwise direction to open the end tool 1100.

Next, the yaw motion is as follows.

When the user rotates the first handle 204 about the rotation shaft 243 while holding the first handle 204, the actuation manipulation part 203 and the yaw manipulation part 202 are yaw-rotated about the rotation shaft 243. That is, when the actuation pulley 262 to which the wire 301 and the wire 305 are fixedly coupled is rotated about the rotation shaft 243, the wire 301 and the wire 305 wound on the pulley 213 and the pulley 214 are moved. Here, one of the wire 301 and the wire 305 is wound on the pulley 213 or the pulley 214, and the other of the wire 301 and the wire 305 may be unwound from the pulley 213 and the pulley 214. Likewise, because the wire 302 and the wire 306 are fixedly coupled to the actuation pulley 262, when the actuation pulley 262 is rotated about the rotation shaft 243, the wire 302 and the wire 306 wound on the pulley 223 and the pulley 224 are moved. Here, one of the wire 302 and the wire 306 is wound on the pulley 223 or the pulley 224, and the other of the wire 302 and the wire 306 is unwound from the pulley 223 or the pulley 224. Here, the wire 301 and the wire 305 connected to the first jaw 1101 and the wire 302 and the wire 306 connected to the second jaw 1102 are wound on the pulley 213 and the pulley 214, and the pulley 223 and the pulley 224 so that the first jaw 1101 and the second jaw 1102 may be rotated in the same direction during yaw rotation. In addition, the rotational force is transmitted to the end tool 1100 via the power transmission part 300, and thus, the yaw motion in which two jaws 1103 of the end tool 1100 are rotated in the same direction is performed.

Here, because the yaw frame 207 connects the first handle 204, the rotation shaft 241, the rotation shaft 242, and the rotation shaft 243, the first handle 204, the yaw manipulation part 202, and the actuation manipulation part 203 are rotated about the rotation shaft 243 along with each other.

Next, the pitch motion is as follows.

When the user rotates the first handle 204 about the rotation shaft 246 while holding the first handle 204, the actuation manipulation part 203, the yaw manipulation part 202, and the pitch manipulation part 201 are pitch-rotated about the rotation shaft 246. That is, when the actuation pulley 262 to which the wire 301 and the wire 305 are fixedly coupled is rotated about the rotation shaft 246, the wire 301 and the wire 305 wound on the pulley 219 and the pulley 220 are moved. Likewise, when the actuation pulley 262 to which the wire 302 and the wire 306 are fixedly coupled is rotated about the rotation shaft 246, the wire 302 and the wire 306 wound on the pulley 229 and the pulley 230 are moved. Here, as described above with reference to FIG. 5, etc., the wire 301 and the wire 305 that are the first jaw wires are moved in the same direction and the wire 302 and the wire 306 that are the second jaw wires are moved in the same direction, and the wire 301, the wire 305, the wire 302, and the wire 306 that are the jaw wires are respectively wound on the pulley 219, the pulley 220, the pulley 229, and the pulley 230 that are the pitch main pulleys of the manipulation part so that the first jaw 1101 and the second jaw 1102 may be pitch-rotated. In addition, the above rotational force is transmitted to the end tool 1100 via the power transmission part 300, and thus, two jaws 1103 of the end tool 1100 perform the pitch motion.

Here, the pitch frame 208 is connected to the yaw frame 207 and the yaw frame 207 connects the first handle 204, the rotation shaft 241, the rotation shaft 242, and the rotation shaft 243, and thus, when the pitch frame 208 rotates about the rotation shaft 246, the yaw frame 207 connected to the pitch frame 208, the first handle 204, the rotation shaft 241, the rotation shaft 242, and the rotation shaft 243 are moved along with each other. That is, when the pitch manipulation part 201 rotates about the rotation shaft 246, the actuation manipulation part 203 and the yaw manipulation part 202 are rotated along with the pitch manipulation part 201.

In summary, in the surgical instrument 10 according to the embodiment of the present disclosure, the pulleys are formed on respective joint points (an actuation joint, a yaw joint, and a pitch joint), the wires (the first jaw wires or the second jaw wires) are wound on the pulleys, and the rotational manipulations (an actuation rotation, a yaw rotation, and a pitch rotation) of the manipulation part cause the movement of respective wires, and consequently, the desired motion of the end tool 1100 may be induced. Furthermore, auxiliary pulleys may be formed at one side of each of the pulleys, and the wire may not be wound several times around one pulley due to the auxiliary pulleys.

FIG. 5 is a diagram schematically showing a configuration of a pulley and a wire configuring a joint of the surgical instrument 10 of FIG. 1. In FIG. 5, the relay pulleys for changing the passage of the wire regardless of the joint motion are omitted.

Referring to FIG. 5 to FIG. 8, the manipulation part 200 may include the pulley 211, the pulley 212, the pulley 213, the pulley 214, the pulley 215, the pulley 217, the pulley 218, the pulley 219, and the pulley 220 related to the rotation movement of the first jaw 1101.

Also, the manipulation part 200 may include the pulley 221, the pulley 222, the pulley 223, the pulley 224, the pulley 225, the pulley 227, the pulley 228, the pulley 229, and the pulley 230 related to the rotation movement of the second jaw 1102. Also, the manipulation part 200 may include the pulley 262 related to rotation movements of the first jaw and the second jaw. (Because the arrangement and configuration of the respective pulleys in the manipulation part 200 are the same as those of the respective pulleys in the end tool 1100 in principle, and thus, specific indications of reference numerals in the drawings are partially omitted.)

The pulleys 211 and 212 and the pulleys 221 and 222 may be formed to rotate independently of each other about the same rotation shaft 242. Here, the pulley 211 and the pulley 212 may include two pulleys that are formed to face each other and be rotatable independently. Likewise, the pulley 221 and the pulley 222 may include two pulleys that are formed to face each other and independently rotatable, and the two pulleys may have different diameters.

The pulleys 213 and 214 and the pulleys 223 and 224 may be formed to rotate independently of each other about the same rotation shaft 243. Here, the pulley 213 and the pulley 214 and the pulley 223 and the pulley 224 may each include two pulleys that are formed to face each other and independently rotatable.

The pulley 215 and the pulley 225 may be formed to be rotatable independently of each other about the same rotation shaft 244.

The pulleys 217 and 218 and the pulleys 227 and 228 may be formed to rotate independently of each other about the same rotation shaft 245. Here, the pulley 217 and the pulley 218 may have different diameters. Also, the pulley 227 and the pulley 228 may have different diameters.

The pulleys 219 and 220 and the pulleys 229 and 230 may be formed to rotate independently of each other about the same rotation shaft 246.

The wire 301 is wound on the pulley 262 after sequentially passing through the pulley 219, the pulley 217, the pulley 215, the pulley 213, and the pulley 211 of the manipulation part 200, and then is coupled to the pulley 262 via a fastening member 530b. In addition, the wire 305 is coupled to the pulley 262 via a fastening member 530d after sequentially passing through the pulley 220, the pulley 218, the pulley 214, and the pulley 212 of the manipulation part 200. Here, the fastening members 530b and 530d may be directly coupled to the pulley 262, or the fastening members 530b and 530d may be coupled to a separate body member 510 and the body member 510 may be coupled to the pulley 262 so that the fastening members 530b and 530d may be coupled to the pulley 262. Therefore, when the pulley 262 rotates, the wire 301 and the wire 305 are wound around or unwound from the pulley 262, causing the first jaw 1101 to rotate.

The wire 306 is wound on the pulley 262 after sequentially passing through the pulley 229, the pulley 227, the pulley 225, the pulley 223, and the pulley 221 of the manipulation part 200, and then is coupled to the pulley 262 via a fastening member 530a. In addition, the wire 302 is coupled to the pulley 262 via a fastening member 530c after sequentially passing through the pulley 230, the pulley 228, the pulley 224, and the pulley 222 of the manipulation part 200. Here, the fastening members 530a and 530c may be directly coupled to the pulley 262, or the fastening members 530a and 530c may be coupled to the separate body member 510 and the body member 510 may be coupled to the pulley 262 so that the fastening members 530a and 530c may be coupled to the pulley 262. Therefore, when the pulley 262 rotates, the wire 302 and the wire 306 are wound around or unwound from the pulley 262, causing the second jaw 1102 to rotate.

In addition, the fastening members 530a, 530b, 530c, and 530d may be directly coupled to the body member 510, but may be connected to the body member 510 while being accommodated in a bolt member 520 that is described later.

The bolt member and the body member will be described in detail later.

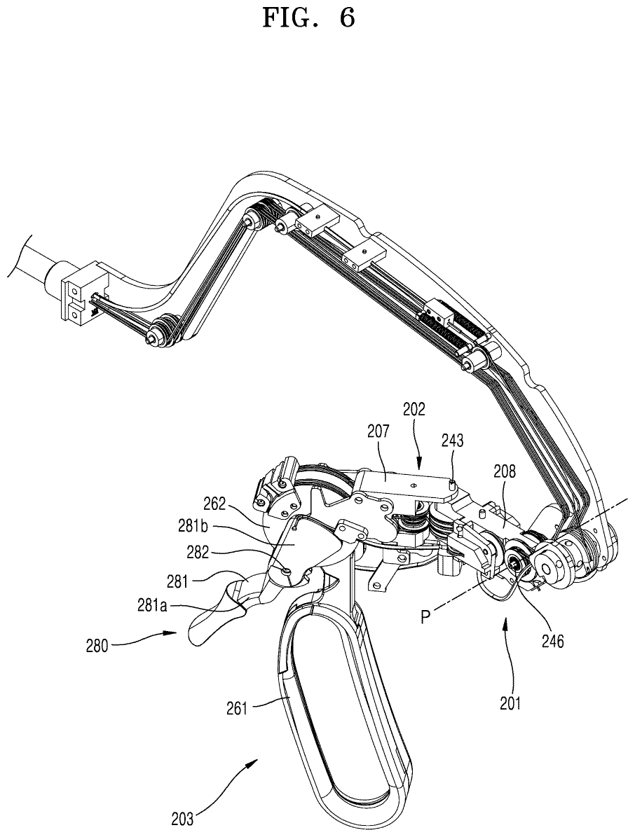

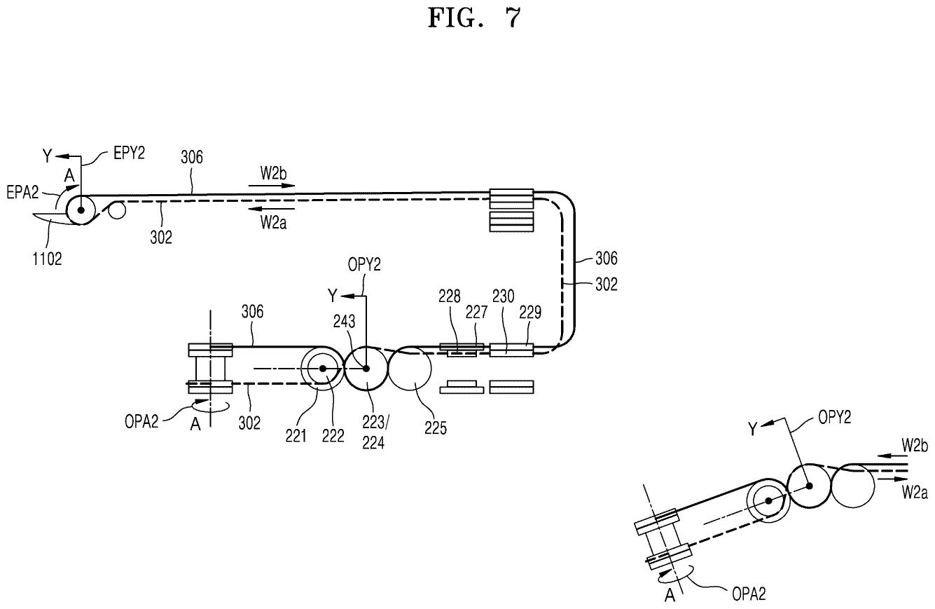

FIGS. 7 and 8 are diagrams showing a configuration of pulleys and wires related to an actuation motion and a yaw motion of the surgical instrument 10 according to an embodiment of the present disclosure of FIG. 1, in detail with respect to each of the first jaw and the second jaw. FIG. 7 is a diagram only showing the pulleys and the wires related to the second jaw, and FIG. 8 is a diagram only showing the pulleys and the wires related to the first jaw. In addition, FIG. 6 is a perspective view illustrating a yaw motion of the surgical instrument of FIG. 1.

First, a wire motion in the actuation motion is described below.

Referring to FIG. 8, when the actuation lever 261 rotates about the rotation shaft 241 in a direction of arrow OPA1, the pulley 262 connected to the actuation lever 261 rotates, and the wire 301 and the wire 305 wound on the pulley 262 are moved in directions W1a and W1b, respectively. Thus, the first jaw 1101 of the end tool 1100 is rotated in a direction of arrow EPA1.

Referring to FIG. 7, when the actuation lever 261 rotates about the rotation shaft 241 in a direction of arrow OPA2, the pulley 262 connected to the actuation lever 261 rotates, and the wire 302 and the wire 306 wound on the pulley 262 are moved in directions W2a and W2b, respectively. Thus, the second jaw 1102 of the end tool 1100 is rotated in a direction of arrow EPA2. Therefore, when the user manipulates the actuation lever 261 in a direction closer to the first handle 204, a motion of the first jaw 1101 and the second jaw 1102 of the end tool being closer to each other is performed.

Next, a wire motion in the yaw motion is described.

First, the rotation shaft 243, the rotation shaft 241, and the rotation shaft 242 are connected via the yaw frame (see 207 of FIG. 3), and thus, the rotation shaft 243, the rotation shaft 241, and the rotation shaft 242 are integrally rotated along with one another.

Referring to FIG. 8, when the first handle 204 is rotated about the rotation shaft 243 in a direction of arrow OPY1, the pulley 262, the pulley 211, the pulley 212, the pulley 213, and the pulley 214 and the wire 301 and the wire 305 wound on the pulleys are entirely rotated about the rotation shaft 243, and consequently, the wire 301 and the wire 305 wound on the pulley 213 and the pulley 214 are moved respectively in directions W1a and W1b, and the first jaw 1101 of the end tool 1100 is rotated in the direction of arrow EPY1.

Referring to FIG. 7, when the first handle 204 is rotated about the rotation shaft 243 in a direction of arrow OPY2, the pulley 262, the pulley 221, the pulley 222, the pulley 223, and the pulley 224 and the wire 302 and the wire 306 wound on the pulleys are entirely rotated about the rotation shaft 243, and consequently, the wire 302 and the wire 306 wound on the pulley 223 and the pulley 224 are moved respectively in directions opposite to W2a and W2b, and the first jaw 1101 of the end tool 1100 is rotated in the direction of arrow EPY2.

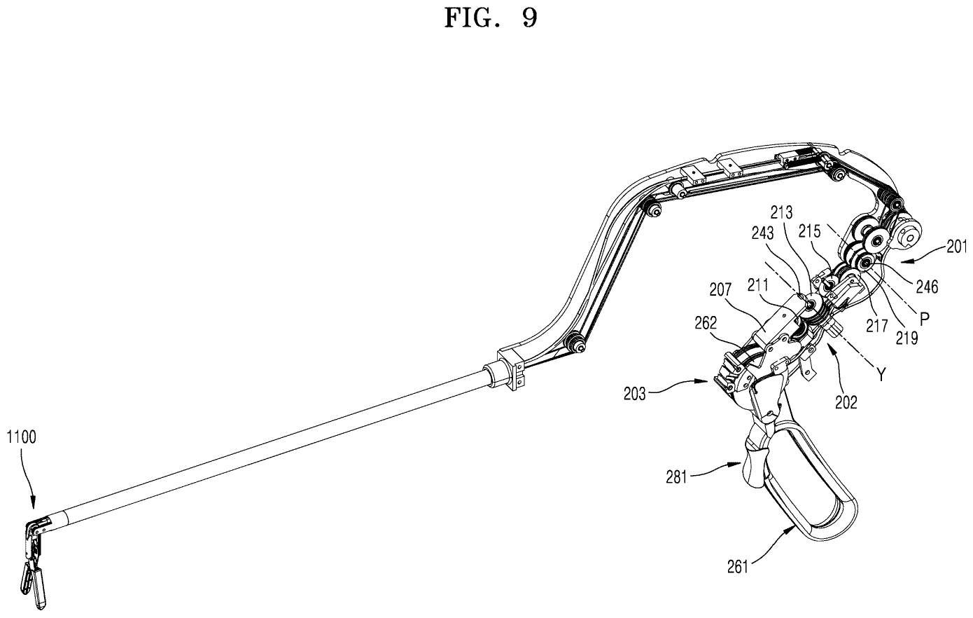

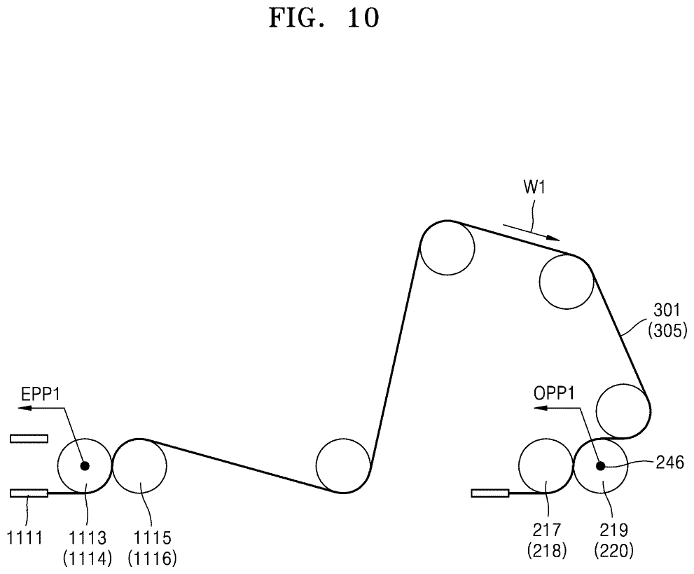

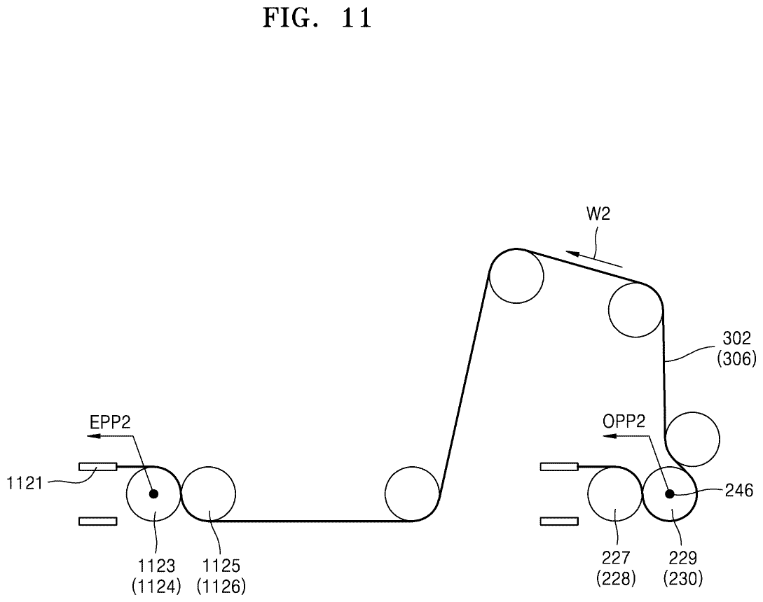

FIGS. 10 and 11 are diagrams showing a configuration of pulleys and wires related to a pitch motion of the surgical instrument 10 according to the embodiment of the present disclosure shown in FIG. 1, in detail with respect to each of the first jaw and the second jaw. FIG. 11 is a diagram only showing the pulleys and the wires related to the second jaw, and FIG. 10 is a diagram only showing the pulleys and the wires related to the first jaw. As shown in FIG. 1, etc., two pulleys are related to the pitch motion, and both strands of each wire are wound along the same path, and FIG. 10 shows the strands as one single line. FIG. 9 is a perspective view illustrating the pitch motion of the surgical instrument of FIG. 1.

Further referring to FIG. 10, when the first handle 204 is rotated about the rotation shaft 246 in a direction of arrow OPP1, the pulley 262, the pulley 217, the pulley 219, etc. and the wire 301, etc. wound on the pulleys are entirely rotated about the rotation shaft 246. Here, the wire 301 and the wire 305 that are the first jaw wires are wound on the upper sides of the pulley 219 and the pulley 220, and thus are moved in a direction of arrow W1. Consequently, the first jaw 1101 of the end tool 1100 is rotated in the direction of arrow EPP1.

Referring to FIG. 11, when the first handle 204 is rotated about the rotation shaft 246 in a direction of arrow OPP2, the pulley 262, the pulley 227, the pulley 229, etc. and the wire 302, etc. wound on the pulleys are entirely rotated about the rotation shaft 246. Here, the wire 302 and the wire 306 that are the second jaw wires are wound on the lower sides of the pulley 229 and the pulley 230, and thus are moved in a direction of arrow W2. Consequently, the second jaw 1102 of the end tool 1100 is rotated in the direction of arrow EPP2.

Therefore, the actuation manipulation, the yaw manipulation, and the pitch manipulation may be performed independently of one another.

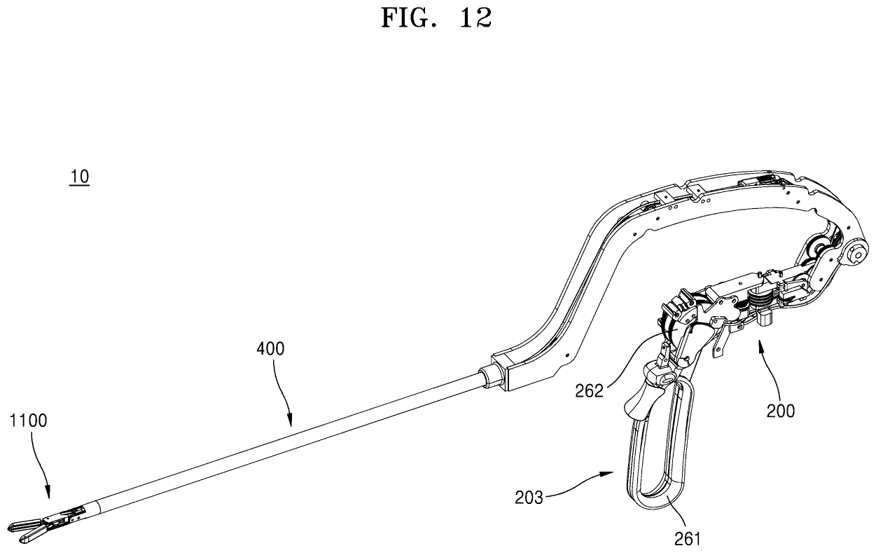

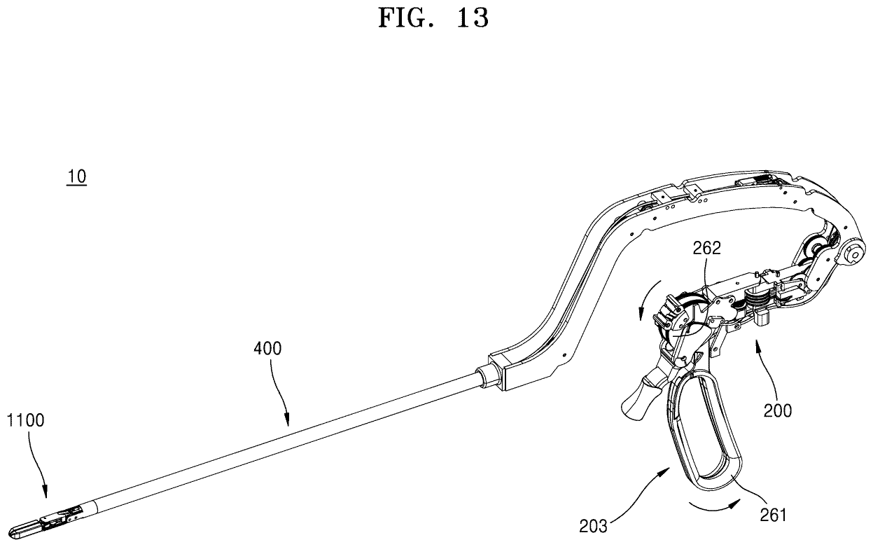

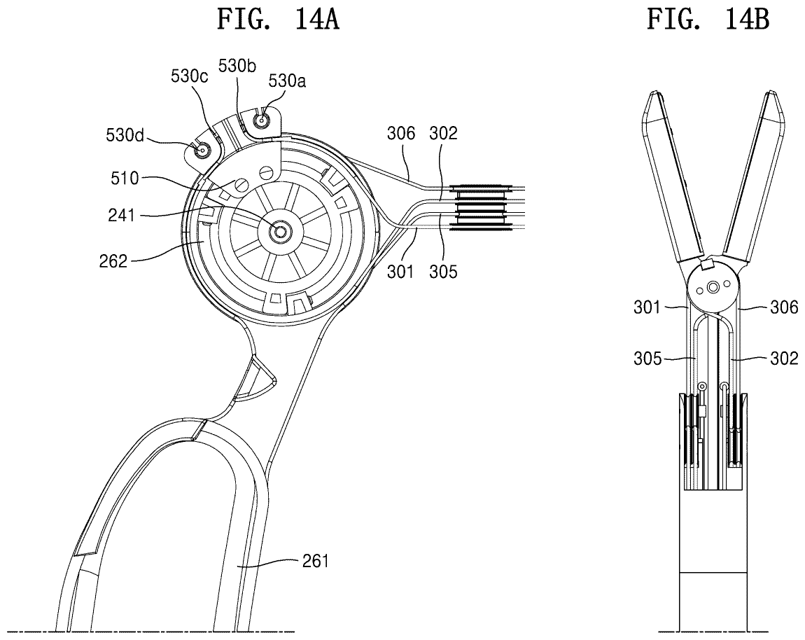

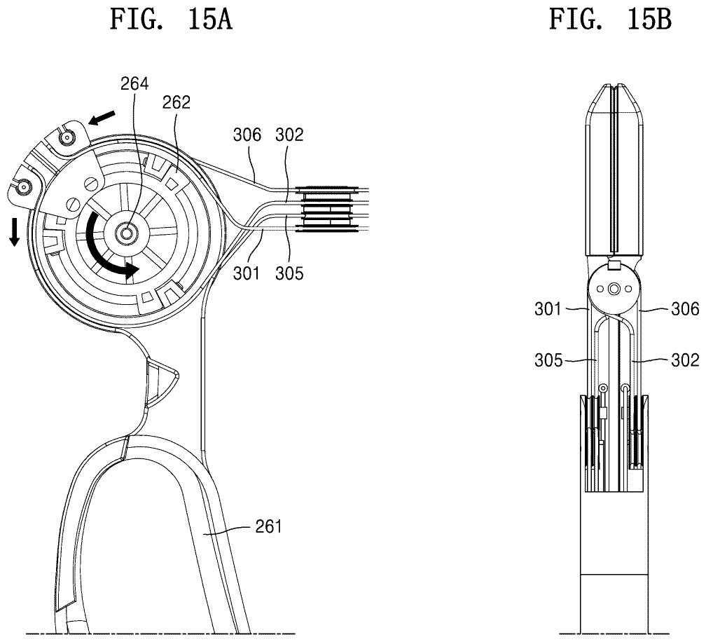

FIGS. 12 and 13 are perspective views showing an actuation lever motion of the surgical instrument of FIG. 1, and FIGS. 14A and 14B, and 15A and 15B are diagrams showing the movement of the wire during the actuation lever motion of the surgical instrument of FIG. 1.

FIG. 12 shows a status in which a case of the surgical instrument of FIG. 1 is removed and the actuation lever does not operate, and FIG. 13 shows a status in which the case of the surgical instrument of FIG. 1 is removed and the actuation lever operates.

Referring to FIGS. 12 and 13, in the surgical instrument 10 according to an embodiment of the present disclosure, the actuation pulley 262 may be rotated by pulling the actuation lever 261 toward the first handle 204 while the first handle 204 is gripped by the palm and the fingers are inserted in the actuation lever 261. That is, the actuation motion may be performed by manipulating one lever.

FIG. 14A is a side view showing wires on the actuation pulley 262 in the surgical instrument of FIG. 1, and FIG. 14B is a plan view showing the wires in the end tool 1100. FIG. 15A is a side view showing the movement of wires on the actuation pulley 262 during the actuation lever motion, and FIG. 15B is a plan view showing the movement of the wires in the end tool 1100.

Referring to FIGS. 5, 14, and 15, in the surgical instrument 10 according to the embodiment of the present disclosure, the wire 301 and the wire 305 that are the first jaw wires and the wire 302 and the wire 306 that are the second jaw wires are all coupled to one actuation pulley 262, and thus, the arrangement of each wire is appropriately configured to differentiate the movement of each wire only with the rotation of one pulley. That is, the first jaw 1101 and the second jaw 1102 may be rotated in different directions through the rotation of the actuation pulley 262 in a certain direction due to the actuation lever 261. In other words, the first jaw 1101 and the second jaw 1102 may perform opening/closing motions according to the rotation of the actuation pulley 262.

In detail, the wire 301 and the wire 305 that are the first jaw wires may be wound on the actuation pulley 262 in opposite directions. For example, as shown in FIG. 5, etc., the wire 301 may be wound on the actuation pulley 262 in the counter-clockwise direction and the wire 305 may be wound on the actuation pulley 262 in the clockwise direction. Likewise, the wire 302 and the wire 306 that are the second jaw wires may be wound on the actuation pulley 262 in opposite directions. For example, as shown in FIG. 5, the wire 302 may be wound on the actuation pulley 262 in the clockwise direction and the wire 306 may be wound on the actuation pulley 262 in the counter-clockwise direction.

Here, when the actuation pulley 262 rotates in the counter-clockwise direction, the wire 301 is wound on the actuation pulley 262 and the wire 305 is unwound from the actuation pulley 262. Accordingly, the wire 301 is unwound from and the wire 305 is wound on the first jaw pulley 1111 of the end tool 1100, and thus, the first jaw pulley 1111 of the end tool is rotated in the counter-clockwise direction.

Also, when the actuation pulley 262 rotates in the counter-clockwise direction, the wire 306 is wound on the actuation pulley 262 and the wire 302 is unwound from the actuation pulley 262. Accordingly, the wire 306 is unwound from and the wire 302 is wound on the second jaw pulley 1121 of the end tool 1100, and thus, the second jaw pulley 1121 of the end tool is rotated in the clockwise direction.

Likewise, when the actuation pulley 262 rotates in the clockwise direction, the first jaw pulley 1111 is rotated in the clockwise direction and the second jaw pulley 1121 is rotated in the counter-clockwise direction.

Therefore, when the actuation pulley 262 rotates, the first jaw pulley 1111 and the second jaw pulley 1121 rotate in the opposite directions from each other, and thus, the first jaw and the second jaw of the end tool 1100 are opened or closed.

Hereinafter, a tension control unit according to an embodiment of the present disclosure is described in detail below.

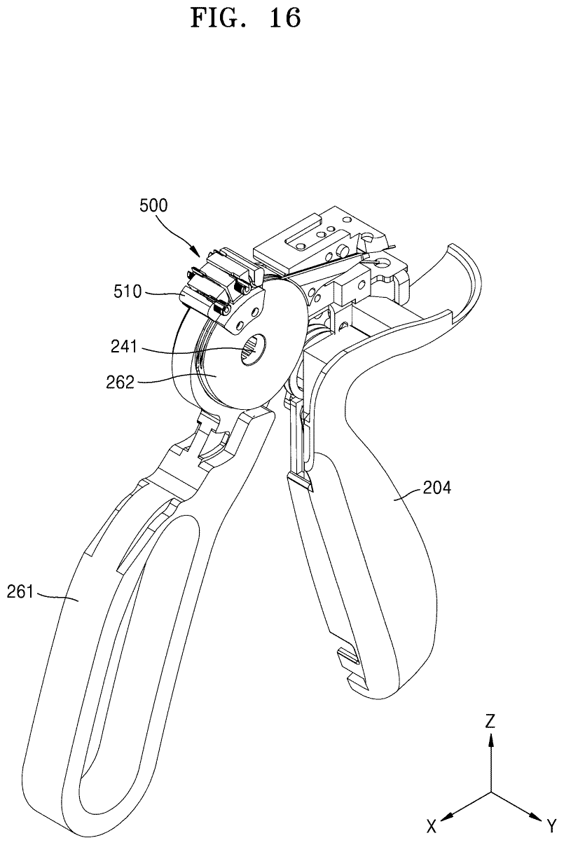

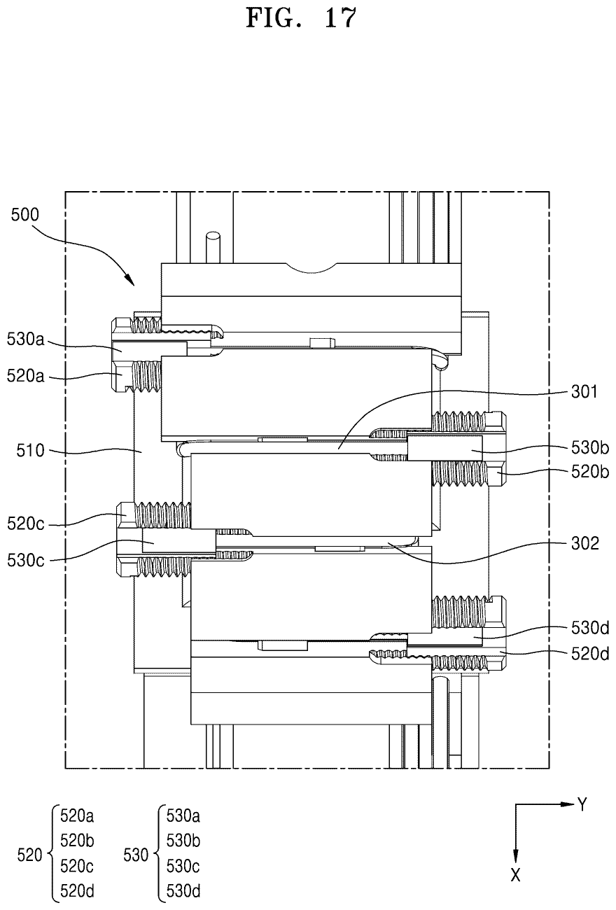

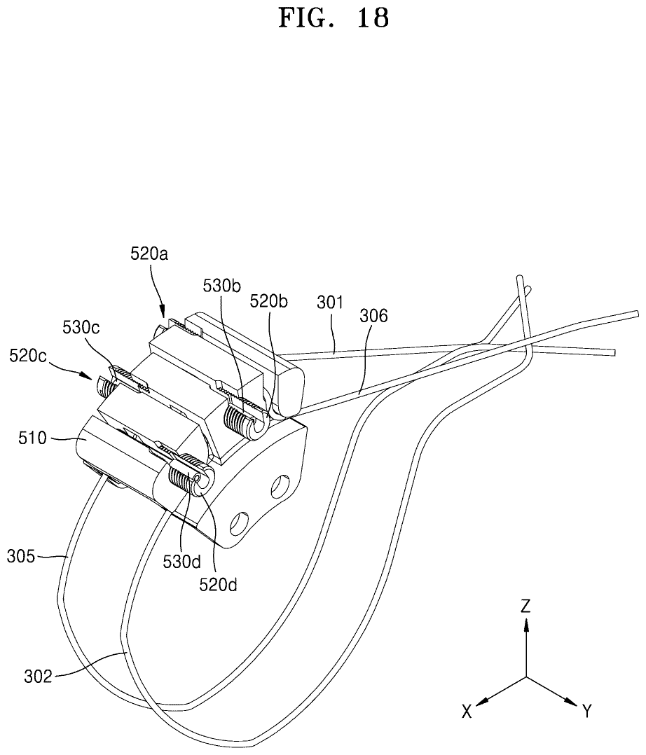

FIG. 16 is a perspective view illustrating a manipulation part including a tension control unit according to an embodiment of the present disclosure. FIG. 17 is a plan view showing the tension control unit of FIG. 16, and FIG. 18 is a perspective view showing the tension control unit and the wires of FIG. 16.

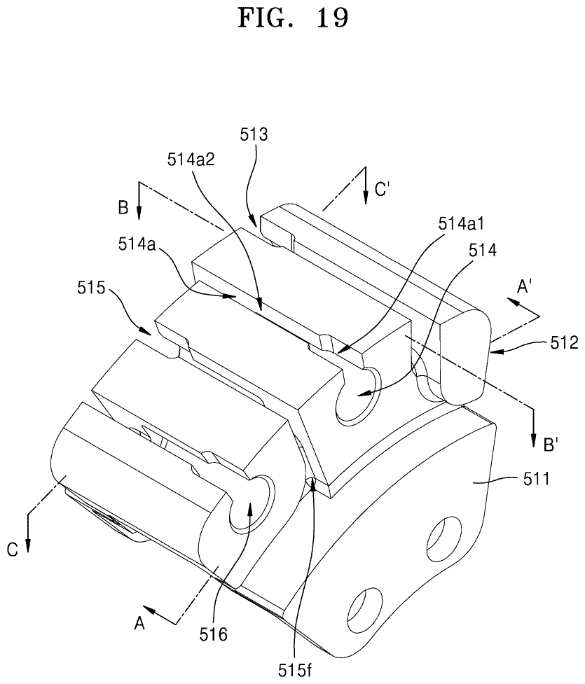

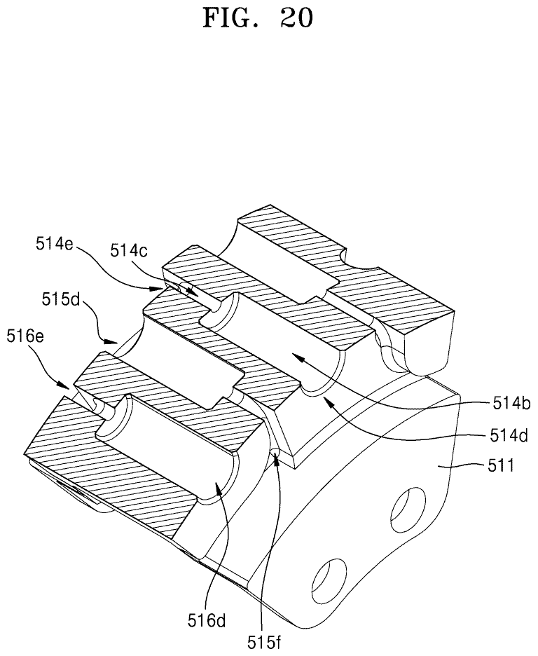

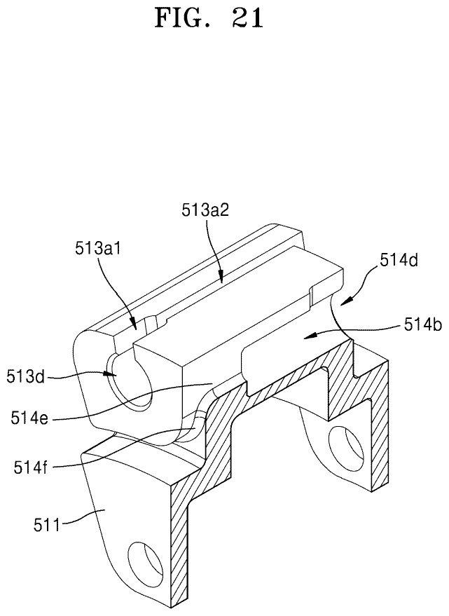

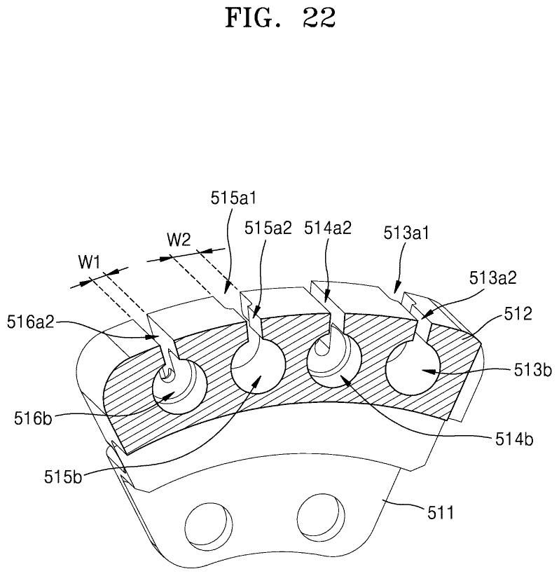

FIG. 19 is a perspective view of a body member in the tension control unit of FIG. 16. FIG. 20 is a cross-sectional view taken along line A-A′ of FIG. 19, FIG. 21 is a cross-sectional view taken along line B-B′ of FIG. 19, and FIG. 22 is a cross-sectional view taken along line C-C′ of FIG. 19.

Referring to FIGS. 16 and 17, a tension control unit 500 according to the embodiment of the present disclosure may include a body member 510, a bolt member 520, and a fastening member 530.

The body member 510 is disposed at one side of the actuation pulley 262 of the manipulation part and may connect the wire and the actuation pulley 262 to each other.

In detail, the body member 510 is a part to which the wires wound on the actuation pulley 262 are fixed, and may be directly coupled to the wires or may be coupled to the wires via the bolt member 520 that is described later.

The tension control unit 500 according to the embodiment of the present disclosure is formed so that the wire is coupled to the body member 510 via the bolt member 520.

The body member 510 may be spaced apart from the rotation shaft 241 of the actuation pulley 262 and arranged at the edge of the actuation pulley 262. In addition, the body member 510 is fixedly coupled to the actuation pulley 262 and may be rotated about the rotation shaft 241 along with the actuation pulley 262.

In addition, according to the embodiment of the present disclosure, the tension control unit 500 is coupled to the actuation pulley 262, but the present disclosure is not limited thereto, and the tension control unit 500 may be arranged on the manipulation part 200, the connection part 400, and the end tool 1100, etc. in order to adjust the tension of the wire in the surgical instrument.

Referring to FIGS. 19 to 23, the body member 510 may include a pulley coupling part 511 that is coupled to the actuation pulley 262, and a wire coupling part 512 to which the wire is coupled.

Also, the body member 510 may form a fastening hole in which the bolt member 520 and the wire are inserted. In detail, the fastening hole may be formed in the wire coupling part 512. In detail, the fastening hole may be formed in a direction perpendicular to a virtual plane on which the actuation pulley 262 rotates. From another perspective, the fastening hole may be formed parallel to the rotation shaft 241 of the actuation pulley 262. However, the direction in which the fastening hole is formed is not limited thereto, but the fastening hole may be formed in various directions and various structures according to the arrangement of the wire.

Also, a plurality of fastening holes may be formed in the body member 510 along the circumference of the actuation pulley 262. In detail, the body member 510 may have the fastening holes, the number of which corresponds to the number of wires coupled to the actuation pulley 262.

Referring to FIG. 17, etc., the body member 510 according to the embodiment of the present disclosure has four fastening holes corresponding to four wires, so that the four wires may be coupled thereto. For example, a fastening hole 513, a fastening hole 514, a fastening hole 515, and a fastening hole 516 may be sequentially formed in the wire coupling part 512. The fastening hole 513 may correspond to the wire 301, the fastening hole 514 may correspond to the wire 306, the fastening hole 515 may correspond to the wire 302, and the fastening hole 516 may correspond to the wire 305.

Hereinafter, a shape and structure of the fastening hole are described based on one fastening hole 514 for convenience of description, but the fastening holes 513, 515, and 516 may be the same as the fastening hole 514 within the corresponding range.

Here, the fastening hole 514 may be formed to pass from one side surface to the other side surface of the body member 510. In detail, the body member 510 may have the opening of the fastening hole 514 in one side surface based on a virtual plane on which the actuation pulley 262 rotates. In addition, the fastening hole 514 is formed in the hole shape extending from the opening and passing through the body member 510, and may form an exit portion in the other side surface of the body member 510.

In addition, the fastening hole 514 may include a first region 514b that may accommodate the wire and the bolt member 520, and a second region 514c that may accommodate the wire. Here, the first region 514b may include a region having an inner diameter that is greater than that of the second region 514c.

For example, the first region 514b may have the inner diameter greater than the second region 514c, in at least some part thereof. Alternatively, the first region 514b may have an inner diameter that is greater than that of the second region 514c.

That is, the first region 514b may form a space corresponding to the bolt member 520 so that the bolt member 520 may be accommodated therein, and the second region 514c may form a space in which the wire may be accommodated. In detail, the first region 514b may have an inner diameter corresponding to an outer diameter of the bolt member 520.

Here, the first region 514b may be formed to have a length corresponding to that of the bolt member 520, but the present disclosure is not limited thereto. The length of the first region 514b may be less than that of the bolt member 520 or may be greater than the bolt member 520. In addition, the first region 514b may have the length greater than that of the second region 514c.

In addition, the fastening hole 514 may have a screw thread (not shown) having a preset pitch formed on the inner surface thereof. For example, the screw thread having a preset pitch may be formed on the inner surface of the first region 514b so as to correspond to a screw thread of the bolt member 520.

The body member 510 may have an opening 514d of the first region in one side surface thereof and an opening 514e of the second region in the other side surface thereof. In other words, the body member 510 may have an entrance of the first region 514b accommodating the bolt member 520 in one side surface thereof, and may have an entrance of the second region 514c accommodating the wire in the other side surface thereof. From another perspective, the body member 510 may have an entrance of the fastening hole 514 in one side surface and an exit of the fastening hole 514 in the other side surface.

Referring back to FIG. 20, the opening of the first region is formed in the other side surface of the body member 510 and the opening of the second region may be formed in one side surface of the body member 510.

For example, the opening 514d of the first region of the fastening hole 514 and an opening 516d of the first region of the fastening hole 516 may be formed in one side surface of the body member 510, and the opening 514e of the second region of the fastening hole 514 and an opening 516e of the second region of the fastening hole 516 may be formed in the other side surface of the body member 510.

In another embodiment, an opening 513d of the first region of the fastening hole 513 and an opening 515d of the first region of the fastening hole 515 may be formed in the other side surface of the body member 510, and an opening 513e of the second region of the fastening hole 513 and an opening 515e of the second region of the fastening hole 515 may be formed in the one side surface of the body member 510. As described above, space utilization may be improved by forming the openings in the first region of each fastening hole in one side or the other side of the body member 510, respectively. That is, in the tension control unit 500 according to the embodiment of the present disclosure, the bolt member 520 may be easily inserted in the body member 510 and the wire fastened with the body member 510 may be easily arranged.

In addition, referring back to FIG. 19, the fastening hole 514 may have a slit 514a formed to have one open side in a direction parallel to the lengthwise direction thereof. That is, the fastening hole 514 may have a structure in which a side surface of a through hole is open. In detail, the slit 514a may be formed from the opening 514d of the first region of the fastening hole 514 to the opening 514e of the second region of the fastening hole 514.

Here, the slit 514a may be wider than a width of the wire and narrower than a width of the bolt member 520. Accordingly, the wire may pass through the slit 514a, but the bolt member 520 may not pass through the slit 514a.

In addition, the slit 514a may have regions with different widths. In detail, the slit 514a may be divided into a first slit region 514a1 and a second slit region 514a2. For example, the first slit region 514a1 may be formed in the first region 514b of the fastening hole 514. In addition, the first slit region 514al may have a width greater than that of the second slit region 514a2. In detail, the first slit region 514al may be formed to have a width through which the fastening member 530 that is described later may pass. Therefore, the tension control unit 500 according to the embodiment of the present disclosure may easily fasten the wire to which the fastening member 530 is coupled with the body member 510.

In addition, the opening 514e of the second region of the fastening hole 514 may include a guide part 514f. Here, the guide part 514f may be a part that guides the wire. In detail, the guide part 514f may guide the wire from the actuation pulley 262 to be arranged along one side surface of the body member 510 and the wire to be inserted into the second region 514c of the fastening hole 514. The guide part 514f may have a curved section on which the wire is arranged so that no strain is applied to the wire even when the wire has a tension.

The guide part 514f is a part extending from the second region 514c and may be formed between the wire coupling part 512 and the pulley coupling part 511. For example, a guide part 515f extending from the opening 515e of the second region of the fastening hole 515 may be formed to pass through a lower end of the wire coupling part 512, in which the opening 516d of the first region of the fastening hole 516 is formed, and the guide part 514f extending from the opening 514e of the second region of the fastening hole 514 may be formed to pass through the lower end of the wire coupling part 512, in which the opening 513d of the first region of the fastening hole 513 is formed.

Hereinafter, the bolt member 520 is described in detail.

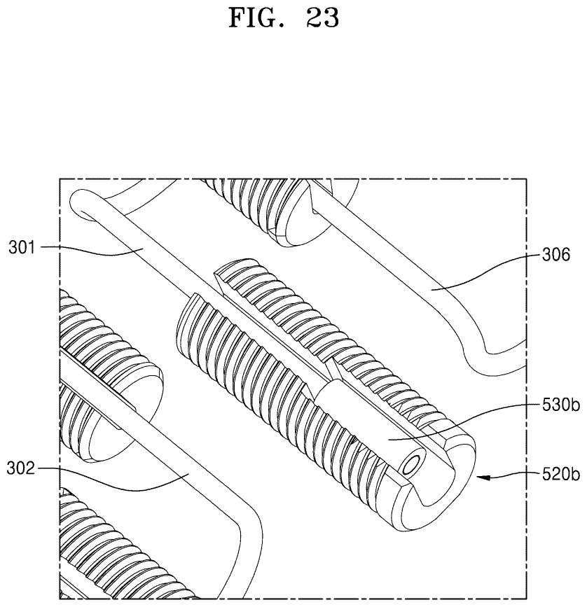

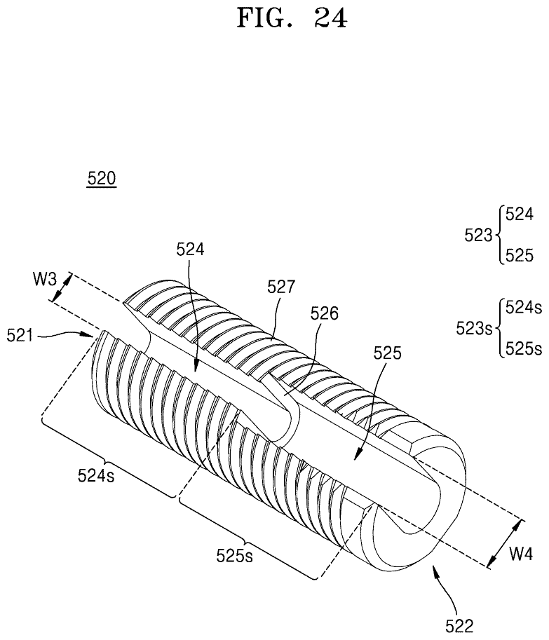

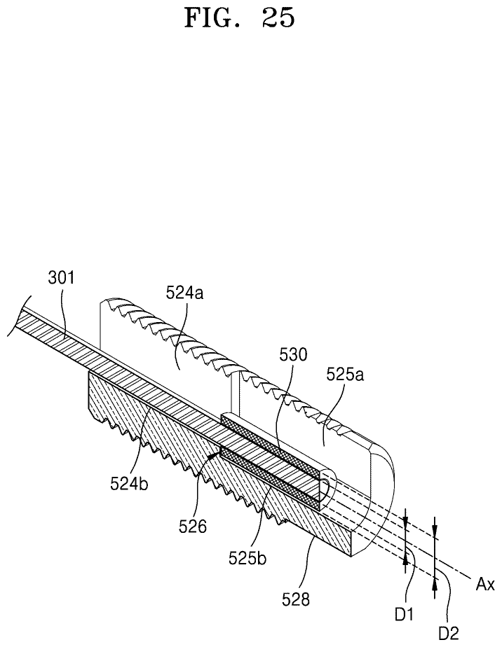

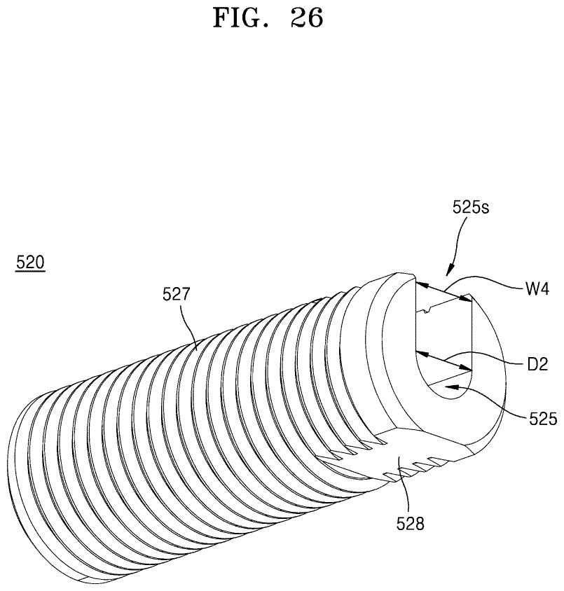

FIG. 23 is a perspective view of the bolt member 520 and wires of FIG. 17. FIG. 24 is a perspective view showing the bolt member 520 of FIG. 23, from which the wire is removed, FIG. 25 is a side-sectional view of the bolt member 520 of FIG. 23, and FIG. 26 is a perspective view showing the bolt member 520 of FIG. 23.

As described above, the bolt member 520 may be a member inserted into the body member 510. In detail, the bolt member 520 may be classified according to the position where the bolt member is inserted into the fastening hole of the body member 510. For example, a bolt member 520a, a bolt member 520b, a bolt member 520c, and a bolt member 520d may be respectively inserted in the fastening hole 513, the fastening hole 514, the fastening hole 515, and the fastening hole 516.

Hereinafter, for convenience of description, the bolt member 520 is described as follows.

The bolt member 520 may have an accommodation part 523 into which the wire and fastening member 530 are inserted. Here, the accommodation part 523 may be formed in the lengthwise direction to pass through one side and the other side of the bolt member 520.

The accommodation part 523 may have a slit having one open side in a direction parallel to the lengthwise direction thereof. In other words, it may be appreciated that the accommodation part 523 of the bolt member 520 is formed in a slit shape.

Here, the accommodation part 523 may have regions with different widths. In detail, the accommodation part 523 includes a first accommodation part 524 capable of accommodating the wire, and a second accommodation part 525 capable of accommodating the wire and the fastening member 530, and the second accommodation part 525 may have a region having an inner diameter greater than that of the first accommodation part 524.

For example, in the second accommodation part 525, a part accommodating the fastening member 530 may have a greater width than that of the first accommodation part 524. Alternatively, the second accommodation part 525 may entirely have a greater width than that of the first accommodation part 524. For example, the second accommodation part 525 may be formed to have the width that is sufficient to accommodate the fastening member 530, and the first accommodation part 524 may be formed to have a width corresponding to the thickness of the wire.

In addition, the bolt member 520 may have a stepped part 526 at a boundary between the second accommodation part 525 and the first accommodation part 524. Here, the stepped part 526 may be a part formed in a stair shape between the second accommodation part 525 and the first accommodation part 524. Because the outer diameter of the fastening member 530 is greater than that of the wire, the fastening member 530 may be stopped by the stepped part 526. Accordingly, the fastening member 530 may be restricted from moving toward the first accommodation part 524 due to the stepped part 526. From another perspective, when the wire is pulled and tension is generated, the fastening member 530 is stopped by the stepped part 526 so as to prevent the wire from moving. A force may be exerted between the fastening member 530 and the stepped part 526 at a contacting surface between the fastening member 530 and the stepped part 526.

In addition, the accommodation part 523 may be formed at the center including a rotation shaft of the bolt member 520. Accordingly, the wire may be disposed on the rotation shaft of the bolt member 520 while being accommodated in the accommodation part 523.

The first accommodation part 524 and the second accommodation part 525 may each have a curved bottom surface. In detail, the bottom of the second accommodation part 525 corresponds to the shape of the fastening member 530, and the bottom of the first accommodation part 524 may correspond to the shape of the wire.

In other words, the inner diameter of the second accommodation part 525 may correspond to an outer diameter D2 of the fastening member 530, and the inner diameter of the first accommodation part 524 may correspond to a diameter D1 of the wire.

In addition, a slit 523s of the bolt member 520 may include a first slit section 524s formed in the first accommodation part 524 and a second slit section 525s formed in the second accommodation part 525. Here, the first slit section 524s may be formed to have a width corresponding to the diameter of the wire, and the second slit section 525s may be formed to have a width corresponding to the outer diameter of the fastening member 530.

Therefore, when the fastening member 530 and the wire are inserted in the accommodation part of the bolt member 520, the wire may be inserted through the first slit section 524s and is accommodated in the first accommodation part 524 along a first accommodation part wall surface 524a, and the fastening member 530 may be inserted through the second slit section 525s and may be accommodated in the second accommodation part 525 along a second accommodation part wall surface 525a.

In addition, the bolt member 520 may have a screw thread 527 corresponding to the screw thread of the fastening hole, and the bolt member 520 is fastened with the fastening hole and the position of the bolt member 520 may be adjusted in the fastening hole in the lengthwise direction according to the rotation of the bolt member 520. A method of controlling the tension of the wire by using the bolt member 520 is described later in detail.

In addition, referring to FIG. 26, the bolt member 520 may have a D-cut part 528 that is flat in a region at the end portion thereof. Alternatively, the bolt member 520 may have the D-cut parts 528 formed at opposite sides facing each other at the end portion thereof.

In detail, the D-cut part 528 may be a part forming a flat surface that is parallel to the rotation shaft of the bolt member 520 and may not have the screw thread 527. In addition, the D-cut part 528 may be formed at the side opposite to the open part of the second slit section 525s.

The D-cut part 528 of the bolt member 520 may be a grip surface of the bolt member 520. The bolt member 520 can be stably gripped by forming the D-cut part 528. Therefore, the user may easily rotate the bolt member 520 in the clockwise or counter-clockwise direction. That is, it may be easy to fasten the bolt member 520 by inserting the bolt member 520 into the fastening hole or to remove the bolt member 520.

Hereinafter, a method of controlling the tension of the wire by using the tension control unit according to an embodiment of the present disclosure is described below.

First, the coupling relationship between the wire and the fastening member 530 is described as follows. The fastening member 530 may be fixed to the wire by fitting into the end of the wire and being crimped.

The wire with the fastening member 530 coupled thereto may be inserted into the accommodation part 523 through the slit 523s of the bolt member 520. In detail, the part of the wire, to which the fastening member 530 is coupled, is accommodated in the second accommodation part 525 of the bolt member 520 and the remaining wire part may be accommodated in the first accommodation part 524.

As described above, the bolt member 520 in which the wire and fastening member 530 are inserted may be inserted into the fastening hole of the body member 510. Alternatively, the wire and fastening member 530 may be inserted into the bolt member 520 while the bolt member 520 is inserted into the fastening hole of the body member 510. Here, the wire may be inserted into the fastening hole 514 via the slit (e.g., 514a) of the body member 510.

Even when the bolt member 520 is rotated while the wire and the fastening member 530 are inserted in the bolt member 520, the bolt member 520 may rotate independently of the wire and the fastening member 530. In other words, because the wire and the fastening member 530 are disposed on the rotation shaft of the bolt member 520 but are not integrally coupled to the bolt member 520, the wire and the fastening member 530 may not rotate along with the bolt member 520 according to the rotation of the bolt member 520, but may remain on the rotation shaft of the bolt member 520.

In addition, referring back to FIG. 17, etc., the wire is wound on the actuation pulley 262, and the end portion of the wire may be coupled to the bolt member 520 of the body member 510. Here, the tension may be exerted on the wire to some extent. That is, the bolt member 520 may be pulling the wire to some extent.

Here, the bolt member 520 may adjust the tension of the wire by pulling or releasing the wire due to the rotation of the bolt member 520 in the clockwise direction or the counter-clockwise direction.

In other words, the bolt member 520 is fastened to the fastening hole, but the position of the bolt member may be adjusted in the lengthwise direction in the fastening hole according to the rotation of the bolt member 520, and thus, the tension of the wire may be adjusted by pulling or releasing the wire. Here, the moving degree of the bolt member 520 may be finely adjusted by forming narrower pitch intervals in the screw threads formed in the fastening hole and the bolt member 520.

For example, when the bolt member 520 rotates in the clockwise direction, the bolt member 520 may be further moved inward while being locked in the fastening hole. Here, as the bolt member 520 moved inward in the fastening hole, the force of the bolt member 520 pulling the wire is decreased, and accordingly, the tension applied to the wire may be reduced.

Also, when the bolt member 520 rotates in the counter-clockwise direction, the bolt member 520 may further move toward the opening while being released in the fastening hole. Here, as the bolt member 520 moves outward in the fastening hole, the force of the bolt member 520 pulling the wire is increased, and accordingly, the tension applied to the wire may be increased.

As described above, the tension control unit 500 according to an embodiment of the present disclosure may adjust the tension of the wire by using the bolt member 520, and as such, an interval between the jaws of the end tool 1100 may be adjusted.

Referring back to FIGS. 7 and 8, as described above, the wire 301 and the wire 305 may be connected to the first jaw 1101, and the wire 302 and the wire 306 may be connected to the second jaw 1102. Here, when the tension is applied to the wire 301 and the wire 306, the first jaw 1101 and the second jaw 1102 are close to each other, and when the tension is applied to the wire 302 and the wire 305, the first jaw 1101 and the second jaw 1102 are away from each other.

Therefore, when the bolt member 520a and the bolt member 520b are connected to the wire 306 and the wire 301, respectively, the tension applied to the wire 306 and the wire 301 is increased and the interval between jaws may be reduced when the bolt member 520a and the bolt member 520b are rotated in the counter-clockwise direction.

In addition, when the bolt member 520c and the bolt member 520d are connected to the wire 302 and the wire 305, respectively, the tension applied to the wire 302 and the wire 305 is increased and the interval between jaws may be increased when the bolt member 520c and the bolt member 520d are rotated in the counter-clockwise direction.

However, the connecting relationship between each bolt member 520 and the wire is not limited to the above example, and the bolt member 520 and the wire may be connected and configured in various combinations.

As described above, the tension control unit 500 according to the embodiment of the present disclosure may adjust the interval between jaws of the end tool 1100 and adjust the grip force of the end tool 1100 by adjusting the tension of the wire.

In addition, the surgical instrument 10 to which the tension control unit 500 according to an embodiment of the present disclosure is applied may adjust the tension even when the wire is assembled, and a constant functional quality of the end tool 1100 may be set before a casing process in the assembling processes.

Also, in a modified example of the embodiment of the present disclosure, a bolt member manipulation part (not shown) that may be manipulated from the outside of the case of the surgical instrument may be provided. That is, because the bolt member manipulation part is connected to the bolt member 520 and protrudes from the outside of the case, the user may adjust the tension of the wire by manipulating the bolt member 520 via the bolt member manipulation part.

A tension control technique according to the related art occupies a relatively large volume in the mechanical part and affects the increase in the weight of the product. However, the tension control unit according to an embodiment of the present disclosure has reduced volume and weight, and thus, the total weight of the product may be reduced.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the scope of the present disclosure is defined not by the detailed description of the present disclosure but by the appended claims, and all differences within the scope will be construed as being included in the present disclosure.

According to the present disclosure, the volume and weight of the surgical instrument may be reduced while adjusting the grip force of the end tool and the interval between jaws by adjusting the tension of the wire in the surgical instrument.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A tension control unit for adjusting a tension of a wire in a surgical instrument, the tension control unit comprising:
a body member arranged at one side of an actuation pulley of a manipulation part and connecting the wire to the actuation pulley;
a fastening member coupled to one end of the wire; and
a bolt member accommodating the fastening member therein and coupled to the body member,
wherein the bolt member adjusts the tension of the wire by pulling or releasing the wire according to rotation thereof in a clockwise direction or a counter-clockwise direction.

2. The tension control unit of claim 1, wherein
the body member has a fastening hole formed therein, in which the bolt member and the wire are inserted, and
the fastening hole is formed to pass from one surface to the other surface of the body member.

3. The tension control unit of claim 2, wherein the fastening hole has a slit having one open side formed in a direction parallel to a lengthwise direction thereof.

4. The tension control unit of claim 3, wherein a width of the slit is greater than a width of the wire and less than a width of the bolt member.

5. The tension control unit of claim 2, wherein the fastening hole includes
a first region capable of accommodating the wire and the bolt member, and
a second region capable of accommodating the wire, and
the first region has a region having an inner diameter that is greater than an inner diameter of the second region.

6. The tension control unit of claim 5, wherein the body member has an opening of the first region formed in one surface thereof and an opening of the second region formed in the other surface thereof.

7. The tension control unit of claim 1, wherein the bolt member has an accommodation part in which the wire and the fastening member are inserted, and
the accommodation part is formed in a lengthwise direction of the bolt member while passing from one side to the other side of the bolt member.

8. The tension control unit of claim 7, wherein the accommodation part includes:
a first accommodation part capable of accommodating the wire; and
a second accommodation part capable of accommodating the wire and the fastening member, and
the second accommodation part has a region having an inner diameter that is greater than an inner diameter of the first accommodation part.

9. The tension control unit of claim 8, wherein the bolt member has a stepped part formed on a boundary between the first accommodation part and the second accommodation part, and
the fastening member is restricted from moving toward the second accommodation part due to the stepped part.

10. The tension control unit of claim 7, wherein the accommodation part has a slit having one open side formed in a direction parallel to a lengthwise direction thereof.

11. The tension control unit of claim 1, wherein the bolt member is rotatable independently of the wire and the fastening member.

12. The tension control unit of claim 2, wherein
the fastening hole has a screw thread having a preset pitch formed on an inner surface thereof and the bolt member has a screw thread formed on an outer circumference thereof and corresponding to the screw thread of the fastening hole, and
the bolt member is fastened with the fastening hole and a position of the bolt member is adjustable in a lengthwise direction within the fastening hole according to the rotation of the bolt member.

13. The tension control unit of claim 12, wherein the bolt member has a D-cut part that is flat and formed in a part of the end portion thereof.

14. A surgical instrument comprising:
an end tool including one or more rotatable jaws and configured to be rotatable in two or more directions;
a manipulation part configured to control rotation of the end tool in the two or more directions;
a power transmission part comprising a wire connected to the manipulation part and transmitting rotation of the manipulation part to the jaws; and
a connection part having one end portion to which the end tool is coupled and the other end portion to which the manipulation part is coupled, and connecting the manipulation part to the end tool,
wherein the manipulation part comprises:
an actuation manipulation part comprising an actuation pulley rotating about one shaft and controlling an actuation motion of the end tool; and
a tension control unit adjusting a tension of the wire,
the tension control unit comprises:
a body member disposed at one side of the actuation pulley and connecting the wire to the actuation pulley;
a fastening member coupled to one end of the wire; and
a bolt member accommodating the fastening member therein and coupled to the body member, and
the bolt member adjusts the tension of the wire by pulling or releasing the wire according to rotation thereof in a clockwise direction or a counter-clockwise direction.

15. The surgical instrument of claim 14, wherein
the body member has a fastening hole formed therein, in which the bolt member and the wire are inserted, and
the fastening hole is formed to pass from one surface to the other surface of the body member.

16. The surgical instrument of claim 15, wherein the fastening hole has a slit having one open side formed in a direction parallel to a lengthwise direction thereof.

17. The surgical instrument of claim 15, wherein the fastening hole includes
a first region capable of accommodating the wire and the bolt member, and
a second region capable of accommodating the wire, and
the first region has a region having an inner diameter that is greater than an inner diameter of the second region.

18. The surgical instrument of claim 14, wherein
the bolt member has an accommodation part in which the wire and the fastening member are inserted, and
the accommodation part is formed in a lengthwise direction of the bolt member while passing from one side to the other side of the bolt member.

19. The surgical instrument of claim 18, wherein the accommodation part includes:
a first accommodation part capable of accommodating the wire; and
a second accommodation part capable of accommodating the wire and the fastening member, and
the second accommodation part has a region having an inner diameter that is greater than an inner diameter of the first accommodation part.

20. The surgical instrument of claim 14, wherein the bolt member is rotatable independently of the wire and the fastening member.
